(19) United States
(12) Patent Application Publication
(10) Pub. No.: US 2023/0372318 A1
(43) Pub. Date: Nov. 23, 2023

(54) METHODS FOR TREATING A SUBJECT HAVING MULTIPLE SYSTEM ATROPHY

(71) Applicant: Theravance Biopharma R&D IP, LLC, South San Francisco, CA (US)

(72) Inventors: Ross VICKERY, San Carlos, CA (US); David L. Bourdet, Millbrae, CA (US); Tadhg Guerin, Dublin (IE); Lucy Norcliffe-Kaufmann, Brooklyn, NY (US); Beiyao Zheng, Palo Alto, CA (US)

(21) Appl. No.: 18/127,570

(22) Filed: Mar. 28, 2023

Related U.S. Application Data
(60) Provisional application No. 63/324,313, filed on Mar. 28, 2022.

(51) Int. Cl. A61K 31/451; A61P 9/02

(52) U.S. Cl. CPC A61K 31/451; A61P 9/02

(57) ABSTRACT

Disclosed are methods for treating a subject having multiple system atrophy (MSA) using ampreloxetine or a pharmaceutically acceptable salt thereof. The methods disclosed include using ampreloxetine or a pharmaceutically acceptable salt thereof to (i) treat the symptoms of neurogenic orthostatic hypotension in a subject having MSA; (ii) reduce the magnitude of decline in the quality of life a subject having MSA; and/or (iii) increase the level of norepinephrine in a subject having MSA.

### CROSS REFERENCE TO RELATED APPLICATIONS

This application claims the benefit under 35 U.S.C. § 119(e) of U.S. Provisional Application No. 63/324,313 filed Mar. 28, 2022, which is hereby incorporated by reference herein in its entirety.

### BACKGROUND

### Field

This application discloses methods for treating a subject having multiple system atrophy (MSA) using ampreloxetine or a pharmaceutically acceptable salt thereof. The methods disclosed include using ampreloxetine or a pharmaceutically acceptable salt thereof to (i) treat the symptoms of neurogenic orthostatic hypotension in a subject having MSA; (ii) reduce the magnitude of decline in the quality of life a subject having MSA; and/or (iii) increase the level of norepinephrine in a subject having MSA.

### State of the Art

Neurogenic orthostatic hypotension (nOH) is a form of orthostatic hypotension (OH), and is highly associated symptoms for multiple system atrophy (MSA) or Parkinson's Disease (PD). nOH is caused by a central or peripheral neurologic disorder, such as MSA or PD, respectively. Such disorders can cause a deficiency or dysregulation of norepinephrine which is the primary neurotransmitter that regulates blood pressure in response to postural changes (Loavenbruck et al, Curr. Med. Res. Opin., 2015; 31:2095-2104). As a result, the autonomic nervous system fails to properly regulate blood pressure during a postural change and the patient experiences a significant fall in blood pressure resulting in, e.g., dizziness, weakness, fatigue, blurry vision, trouble concentrating, head and neck discomfort or syncope.

Accordingly, one objective of nOH treatment is to increase levels of norepinephrine in patients. One way to increase norepinephrine levels is to administer an agent that generates norepinephrine. For example, droxidopa (L-threo-3-4-dihydroxyphenylserine) is an amino acid that is converted by decarboxylation into norepinephrine in both the central and the peripheral nervous systems thereby increasing levels of norepinephrine (Kaufmann et al., Circulation, 2003; 108:724-728; Kaufmann, Clin. Auton. Res. (2008) 18[Suppl 1]:19-24); and Isaacson et al., Vascular Health and Risk Management, 2014, 10:169-176). Droxidopa is approved in the U.S. for the treatment of orthostatic dizziness, light-headedness, or the “feeling that you are about to black out” in adult patients with symptomatic nOH caused by primary autonomic failure (Parkinson's disease, multiple system atrophy, and pure autonomic failure), dopamine beta-hydroxylase deficiency, and nondiabetic autonomic neuropathy. The main side effect of droxidopa is supine hypertension and its prescribing information includes a black box warning due to this serious side effect. Further, the prescribing information for droxidopa states that effectiveness beyond 2 weeks of treatment has not been established.

Alternatively, norepinephrine levels can be increased in patients by inhibiting the norepinephrine transporter which is responsible for norepinephrine reuptake. For example, atomoxetine is a selective norepinephrine reuptake inhibitor approved in the U.S. for treatment of attention-deficit hyperactivity disorder (ADHD). Atomoxetine has been shown to increase blood pressure in patients with central autonomic failure (Ramirez et al., Hypertension, 2014; 64:1235-40; and Shibao et al., Hypertension, 2007; 50:47-53). Atomoxetine, however, is metabolized primarily through the CYP2D6 enzymatic pathway and therefore, its pharmacokinetic properties are variable depending on whether the subject has reduced CYP2D6 activity (poor metabolizer) or normal CYP2D6 activity (extensive metabolizer) (Ring et al., Drug Metabolism and Distribution, 2002, 30:319-323). Additionally, when used to treat ADHD, atomoxetine is associated with a number of gastrointestinal adverse effects including dry mouth and nausea. Atomoxetine has not been approved for the treatment of nOH. Further, in a recent study, atomoxetine has been shown to be ineffective against nOH in MSA patients, while showing improvement in non-MSA patients. (Urechie et al., Hypertension, 2022; 79[Suppl. 1]: AP063-AP063)

An α1-adrenoceptor agonist, midodrine is the only other FDA-approved drug for the treatment of symptomatic nOH, and other agents used to treat nOH include the synthetic mineralocortioid, fludrocortisone; and the cholinesterase inhibitor, pyridostigmine. The side effects of these agents can include, for midodrine, supine hypertension, paraesthesias (including scalp-tingling), piloerection (goose bumps), and urinary urgency or retention; for fludrocortisone, hypokalemia, headaches, peripheral edema, heart failure and supine hypertension; and for pyridostigmine, abdominal discomfort and urinary urgency.

Accordingly, it would be desirable to have other options available for treating nOH, in particular for MSA patients. In particular, it would be desirable to provide a safe and well-tolerated medication with predictable pharmacokinetic properties for use in the treatment of nOH, and it would be also desirable to provide a medication that is effective for a prolonged period, and effective against various subgroups of patients.

### SUMMARY

It has now been discovered that certain symptoms associated with nOH are surprisingly reduced for a prolonged time when a subject having multiple system atrophy is treated with ampreloxetine. For example, in a subject having multiple system atrophy and symptomatic neurogenic orthostatic hypotension, treatment with ampreloxetine for at least about 22 weeks surprisingly results in a measurable reduction in both (i) the subject's symptoms of dizziness, lightheadedness, feeling faint, or feeling like he or she might black out; and (ii) the subject's overall impression of symptom severity. Moreover, the magnitude of decline in the quality of life of a subject having multiple system atrophy may be reduced when the subject is treated with ampreloxetine for at least about 22 weeks. Surprisingly, it has also been discovered that the norepinephrine level in a subject having multiple system atrophy continues to increase for at least about 8 weeks when the subject is treated daily with ampreloxetine.

Accordingly, the present disclosure provides methods for treating a subject having multiple system atrophy using ampreloxetine or a pharmaceutically acceptable salt thereof, or a pharmaceutical composition comprising ampreloxetine or a pharmaceutical acceptable salt thereof and a pharmaceutically acceptable carrier. The methods disclosed here include but are not limited to using ampreloxetine or a pharmaceutically acceptable salt thereof to (i) treat symptoms of neurogenic orthostatic hypotension in a subject having multiple system atrophy; (ii) reduce the magnitude of decline in the quality of life of a subject having multiple system atrophy; and/or (iii) increase norepinephrine levels in a subject having MSA.

In one aspect, the present disclosure relates to a method for treating symptomatic neurogenic orthostatic hypotension (nOH) in a subject having multiple system atrophy (MSA), the method comprising administering daily to the subject for at least about 8 weeks a pharmaceutical composition comprising a pharmaceutically acceptable carrier and about 10 mg (free base equivalents) of ampreloxetine or a pharmaceutically acceptable salt thereof, wherein the administration results in reduction of at least one of Orthostatic Hypotension Symptom Assessment (OHSA) composite score, Orthostatic Hypotension Daily Activities Scale (OHDAS) item 1 score (standing for a short time), and OHDAS item 3 score (walking for a short time).

In some embodiments, the pharmaceutical composition is administered to the subject orally and/or once per day. In some embodiments, the subject being treated experiences symptoms of neurogenic orthostatic hypotension in the absence of treatment with the pharmaceutical composition as determined using the Orthostatic Hypotension Assessment Scale (OHSA). In some embodiments, the subject has MSA subtype P (MSA-P) or MSA subtype C (MSA-C). In some embodiments, the subject has a sustained reduction in BP of ≥20 mmHg (systolic) or ≥10 mmHg (diastolic) within 3 min of standing as part of orthostatic standing test or being tilted up ≥60° from a supine position as determined by a tilt-table test before the administration. In some embodiments, prior to treating, the subject has score 4 or less on Unified Multiple System Atrophy Rating Scale (UMSARS) Part IV before the administration. In some embodiments, the subject has at least a 4 on the OHSA item 1 score before the administration. In some embodiments, the pharmaceutical composition is administered for at least about 12 weeks. In some embodiments, ampreloxetine is administered as a hydrochloride salt. In some embodiments, the pharmaceutically acceptable carrier comprises one or more of microcrystalline cellulose, lactose and magnesium stearate.

In one aspect, the present disclosure relates to a method for treating symptomatic neurogenic orthostatic hypotension (nOH) in a subject having multiple system atrophy (MSA), the method comprising administering daily to the subject for at least about 22 weeks a pharmaceutical composition comprising a pharmaceutically acceptable carrier and about 10 mg (free base equivalents) of ampreloxetine or a pharmaceutically acceptable salt thereof, wherein:
(a) the subject being treated experiences symptoms of neurogenic orthostatic hypotension in the absence of treatment with the pharmaceutical composition as determined using the Orthostatic Hypotension Assessment Scale (OHSA); and
(b) administration of the pharmaceutical composition daily to the subject for at least about 22 weeks results in a measurable reduction in at least one of (i) the subject's OHSA composite score, (ii) Orthostatic Hypotension Questionnaire (OHQ) composite score, (iii) Orthostatic Hypotension Daily Activity Scale (OHDAS) composite score, or (iv) OHDAS item 1 (standing short time).

In some embodiments, the pharmaceutical composition is administered to the subject orally. In some embodiments, the pharmaceutical composition is administered to the subject once per day. In some embodiments, the supine plasma norepinephrine level in the subject is less than about 350 pg/mL before treatment with the pharmaceutical composition. In some embodiments, the supine plasma norepinephrine level in the subject is greater than about 500 pg/mL after about 8 weeks of treatment with the pharmaceutical composition. In some embodiments, the subject has MSA subtype P (MSA-P). In some embodiments, the subject has been diagnosed with MSA for at least 1.3 years before the administration. In some embodiments, the subject had onset of nOH 1.6 years or more before the administration. In some embodiments, the subject had an OSHA composite score of 5 or greater before the administration. In some embodiments, the subject had an OSHA item 1 score of 7 or greater before the administration. In some embodiments, the subject has a score ≤4 on Unified Multiple System Atrophy Rating Scale (UMSARS) Part IV before the administration. In some embodiments, the subject has a score of 4 on Unified Multiple System Atrophy Rating Scale (UMSARS) Part IV before the administration. In some embodiments, ampreloxetine is administered as a hydrochloride salt. In some embodiments, the pharmaceutical composition is administered for at least about 12 months. In some embodiments, the pharmaceutically acceptable carrier comprises one or more of microcrystalline cellulose, lactose and magnesium stearate.

In one aspect, the present disclosure relates to a method of identifying a subject having multiple system atrophy (MSA) and responsive to ampreloxetine, comprising administering the subject for at least 8 weeks with a pharmaceutical composition comprising a pharmaceutically acceptable carrier and about 10 mg (free base equivalents) of ampreloxetine or a pharmaceutically acceptable salt thereof.

In some embodiments, the pharmaceutical composition is administered for at least 12 weeks. In some embodiments, the method further comprises determining whether the subject shows decrease of at least 2 points in Orthostatic Hypotension Symptom Assessment (OHSA) item 1 score subsequent to the administration. In some embodiments, the pharmaceutical composition is administered to the subject orally. In some embodiments, the pharmaceutical composition is administered to the subject once per day. In some embodiments, the MSA is MSA subtype P (MSA-P) or MSA subtype C (MSA-C).

In one aspect, a method of treating symptomatic neurogenic orthostatic hypotension (nOH) in a subject having multiple system atrophy (MSA), comprising administering to the subject a pharmaceutical composition comprising a pharmaceutically acceptable carrier and ampreloxetine or a pharmaceutically acceptable salt thereof, wherein the subject has:
(a) Orthostatic Hypotension Assessment Scale (OSHA) composite score of 5 or greater before the administration; and/or
(b) OSHA item 1 score of 7 or greater before the administration.

Also provided herein are uses of a pharmaceutical composition comprising a pharmaceutically acceptable carrier and about 10 mg (free base equivalents) of ampreloxetine or a pharmaceutically acceptable salt thereof, for the treatment of symptomatic neurogenic orthostatic hypotension (nOH) in a subject having multiple system atrophy (MSA).

Other aspects and embodiments are disclosed herein.

### BRIEF DESCRIPTION OF THE DRAWINGS

Various aspects of the disclosure are illustrated by reference to the accompanying drawings.

FIG. 1A illustrates the OHSA item 1 score for subjects having multiple system atrophy administered either ampreloxetine or placebo at the end of the 6-week double-blind randomized period.

FIG. 1B illustrates the OHSA composite score for subjects having multiple system atrophy administered either ampreloxetine or placebo at the end of the 6-week double-blind randomized period.

FIG. 2A illustrates the OHDAS composite score for subjects having multiple system atrophy administered either ampreloxetine or placebo at the end of the 6-week double-blind randomized period.

FIG. 2B illustrates the mean change in systolic blood pressure for subjects having multiple system atrophy administered either ampreloxetine or placebo at the end of the 6-week double-blind randomized period.

FIG. 3 illustrates the OHSA and OHDAS composite scores and individual subscores for symptoms and daily activities for subjects having multiple system atrophy administered either ampreloxetine or placebo at the end of the 6-week double-blind randomized period.

FIG. 4 illustrates the EQ-VAS score means for subjects having multiple system atrophy at various weeks of treatment.

FIG. 5 illustrates the mean difference in OHSA composite scores between ampreloxetine and placebo in subgroups of subjects having multiple system atrophy administered either ampreloxetine or placebo at the end of the 6-week double-blind randomized period.

FIG. 6 illustrates the mean difference in OHQ composite scores between ampreloxetine and placebo in subgroups of subjects having multiple system atrophy administered either ampreloxetine or placebo at the end of the 6-week double-blind randomized period.

### DETAILED DESCRIPTION

In various aspects and embodiments, the present application discloses methods for treating a subject having multiple system atrophy using ampreloxetine or a pharmaceutically acceptable salt thereof.

### Definitions

When describing the aspects and embodiments disclosed herein, the following terms have the following meanings unless otherwise indicated.

The singular terms “a,” “an” and “the” include the corresponding plural terms unless the context of use clearly dictates otherwise.

The term “about” means±5 percent of the specified value.

The term “melting point” means the temperature at which the maximum endothermic heat flow is observed by differential scanning calorimetry for the thermal transition that corresponds to the solid-to-liquid phase change.

The term “pharmaceutically acceptable” means acceptable for administration to a subject (e.g., having acceptable safety for the specified usage).

The term “pharmaceutically acceptable salt” means a salt prepared from an acid and a base (including zwitterions) that is acceptable for administration to a subject (e.g., a salt having acceptable safety for a given dosage regime).

The term “subject” means a human subject or patient.

The term “therapeutically effective amount” means an amount sufficient to effect treatment when administered to a subject in need of treatment, e.g., the amount needed to obtain the desired therapeutic effect.

The term “treating” or “treatment” means ameliorating or suppressing the medical condition or disorder being treated; or alleviating the symptoms of the medical condition or disorder.

The term “unit dosage form” or “unit doses” means a physically discrete unit suitable for dosing a subject, i.e., each unit containing a predetermined quantity of a therapeutic agent calculated to produce a therapeutic effect either alone or in combination with one or more additional units. Examples include capsules, tablets and the like.

The term “seated systolic blood pressure” means systolic blood pressure recorded after being seated for 5 minutes and 10 minutes.

The term “standing systolic blood pressure” means systolic blood pressure recorded after standing for 1, 3, 5, and 10 minutes.

All other terms used herein are intended to have their ordinary meaning as understood by persons having ordinary skill in the art to which they pertain.

### Compound of Formula I

Ampreloxetine, or 4-[2-(2,4,6-trifluorophenoxymethyl)phenyl]piperidine, is a norepinephrine reuptake inhibitor having the formula I:

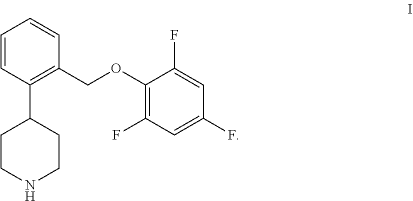 [SMILES: Fc1cc(F)c(OCc2ccccc2C2CCNCC2)c(F)c1]

See, for example, Smith et al., Int J Neuropsychopharmacol. 2015; 18(2)pyu027. Ampreloxetine is also known as TD-9855.

U.S. Pat. Nos. 8,304,432; 8,604,058; 9,162,982; 9,675,599; 10,034,870; 10,306,913; 10,441,579; 10,722,504; and 10,946,007 disclose 4-[2-(2-fluorophenoxymethyl)phenyl]piperidine compounds including ampreloxetine and pharmaceutically acceptable salts thereof. Additionally, U.S. Pat. Nos. 8,304,433; 8,592,596; 9,073,859; 10,226,454; 10,576,073; and 10,946,006 disclose a crystalline hydrochloride salt of ampreloxetine. These patents disclose various uses for ampreloxetine and its salts including treatment of pain disorders, depressive disorders, cognitive disorders, stress urinary incontinence, chronic fatigue syndrome, obesity, vasomotor symptoms associated with menopause, chronic low back pain, osteoarthritis and other disorders.

U.S. Pat. No. 10,238,642 discloses methods for treating neurogenic orthostatic hypotension (nOH) and the symptoms thereof in a human patient having multiple system atrophy (MSA), Parkinson's disease (PD), or pure autonomic failure (PAF) using ampreloxetine and pharmaceutically acceptable salts thereof.

Ampreloxetine has been evaluated in several clinical trials. For example, the safety and tolerability of ampreloxetine were evaluated in a single ascending dose study in healthy subjects at doses ranging from 2 to 50 mg and a multiple ascending dose study in healthy subjects at daily doses of 4, 10, 20, and 40 mg for up to 14 days. In healthy subjects, a single dose up to 50 mg and multiple doses up to 20 mg once per day of ampreloxetine were generally well tolerated.

The safety, tolerability, and efficacy of ampreloxetine were also evaluated in two Phase 2 studies in subjects with attention-deficit hyperactivity disorder and fibromyalgia. Ampreloxetine doses of 5 mg or 20 mg were administered once per day for 6 weeks in both studies and ampreloxetine was generally well tolerated with no clinically significant safety signals.

In these studies, the pharmacokinetic (PK) properties of ampreloxetine were linear with near dose proportional exposure in terms of maximum concentration (Cₘₐₓ) and area under the curve (AUC) for the administered doses. Ampreloxetine has an elimination half-life (t_{1/2}) of approximately 30 to 40 hours achieving steady state by 6 days. Consistent with the elimination half-life, 3 to 4× accumulation of ampreloxetine was observed at steady state. Based on clinical PK studies in healthy subjects, preloxetine is >90% eliminated through metabolism with cytochrome P450 1A2 (CYP1A2) being the primary enzyme responsible for ampreloxetine metabolism. See, e.g., Kanodia et al., “Pharmacokinetics of Ampreloxetine, a Norepinephrine Reuptake Inhibitor, in Healthy Subjects and Adults with Attention-Deficit/Hyperactive Disorder or Fibromyalgia Pain,” Clin Pharmacokinetics, (2021) 60:121-131.

Ampreloxetine was also evaluated in a multicenter, randomized, three part, single-blind (Part A), double-blind, placebo-controlled (Part B), and open-label multiple dose extension (Part C) Phase 2 study in subjects with symptomatic nOH. Part A evaluated the dose response of single, ascending doses of ampreloxetine up to 20 mg. Although no dose response was observed, a numerical trend in increased seated and standing systolic blood pressure (SBP) was observed at the higher doses. An improvement in standing time of approximately 100 seconds was observed at 4 hours after dosing at 10 mg. In Part B, subjects were treated with ampreloxetine (up to 15 mg) in a double-blind, placebo-controlled 1-day inpatient study. Subjects receiving ampreloxetine demonstrated a sustained increase in SBP over baseline following 3 minutes of standing at 4- and 7-hours post-dose. An increase in SBP was not observed in placebo-treated subjects at these time points. See, e.g., Kaufmann et al., Clin Auton Res (2021) 31:699-711; and Kaufmann et al., “A Phase 2, Dose-Escalation Study of Ampreloxetine (TD-9855), a Norepinephrine Reuptake Inhibitor, Given Once-Daily to Treat Neurogenic Orthostatic Hypotension (nOH) in Subjects with Synucleinopathies.” Poster 126 presented at the International Parkinson and Movement Disorder Society; Sep. 22-26, 2019; Nice, France.

Part C of this study evaluated durability of response, safety, and tolerability of ampreloxetine in an open-label phase 2 multicenter study of subjects with nOH. Subjects were treated with oral ampreloxetine (3-20 mg) once-daily for up to 20 weeks with a 4-week follow-up after ampreloxetine treatment withdrawal. The primary efficacy endpoint for Part C was an improvement from baseline of at least 2 points in the Orthostatic Hypotension Symptom Assessment (OHSA) Question 1 (OHSA #1) score. OHSA #1 is a measure of dizziness, lightheadedness, feeling faint, or the sensation of being about to black out. A large proportion of subjects met this endpoint. See, e.g., Kaufmann et al., Clin Auton Res (2021) 31:699-711; and Kaufmann et al., “Efficacy, Durability, and Safety of Ampreloxetine, a Norepinephrine Reuptake Inhibitor, Given Once-Daily to Treat Neurogenic Orthostatic Hypotension (nOH) in Subjects with Primary Autonomic Failure.” Poster 087 presented at the World Congress on Parkinson's Disease and Related Disorders; Jun. 16-19, 2019; Montreal, Canada; and Kaufmann et al., “A Phase 2 study of the Efficacy, Durability, and Safety of Ampreloxetine (TD-9855), a Norepinephrine Reuptake Inhibitor, Given Once-Daily to Treat Neurogenic Orthostatic Hypotension (nOH) in Subjects with Synucleinopathies.” Poster 125 presented at the International Parkinson and Movement Disorder Society; Sep. 22-26, 2019; Nice, France.

More recently, ampreloxetine was evaluated in two Phase 3 studies. The first Phase 3 study was a randomized, double-blind, placebo-controlled, parallel-group, multicenter study designed to evaluate its efficacy, safety, and tolerability in subjects with primary autonomic failures (MSA, PD, or PAF) and symptomatic nOH after 4 weeks of treatment. The primary objective of this study was to evaluate the efficacy of ampreloxetine in subjects with MSA, PD or PAF experiencing symptomatic nOH compared with placebo at 4 weeks as measured by the change from baseline of the OHSA #1 score. After 4 weeks of treatment, this study did not meet the primary endpoint.

The second Phase 3 study was a multi-center, randomized withdrawal study to evaluate the sustained benefit in efficacy and safety of ampreloxetine in subjects with primary autonomic failures (MSA, PD, or PAF) and symptomatic nOH after 22 weeks of treatment. The study consisted of a 16-week open-label (OL) treatment period with ampreloxetine followed by a 6-week double-blind randomized withdrawal period where subjects were administered either ampreloxetine or placebo.

The aspects and embodiments disclosed herein employ ampreloxetine (TD-9855 or 4-[2-(2,4,6-trifluorophenoxy-methyl)phenyl]piperidine) having formula I.

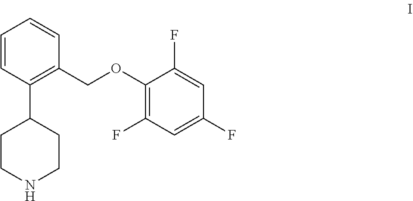 [SMILES: Fc1cc(F)c(OCc2ccccc2C2CCNCC2)c(F)c1]

or a pharmaceutically acceptable salt thereof.

Ampreloxetine and intermediates thereto can be prepared as described in the Examples herein or by the methods and procedures disclosed in U.S. Pat. Nos. 8,304,432; 8,304,433; 8,247,433; and 10,640,467, which are incorporated by reference herein; and related patents.

In some embodiments, ampreloxetine is used in the form of a pharmaceutically acceptable salt. Representative pharmaceutically acceptable salts include salts of the following acids (with the corresponding anion shown in parentheses): acetic (acetate), ascorbic (ascorbate), benzenesulfonic (benzenesulfonate or besylate), benzoic (benzoate), camphorsulfonic (camphorsulfonate), chlortheophylline (chlortheophyllinate), citric (citrate), ethanesulfonic (ethanesulfonate), ethanedisulfonic or edisylic (ethanedisulfonate or edisylate), fumaric (fumarate), gentisic (gentisate), gluconic (gluconate), glucuronic (glucoronate), gluceptic (gluceptate), glutamic (glutamate), hippuric (hippurate), hydrobromic (bromide), hydrochloric (chloride), hydroiodic (iodide), isethionic (isethionate), lactic (lactate), lactobionic (lactobionate), laurylsulfonic (laurylsulfonate), maleic (maleate), malic (malate), mandelic (mandelate), methanesulfonic (methanesulfonate or mesylate), methyl sulfonic (methyl sulfonate), mucic (mucate), naphthalenesulfonic (naphthalenesulfonate or napsylate), naphthalene-1,5-disulfonic (naphthalene-1,5-disulfonate), naphthalene-2,6-disulfonic (naphthalene-2,6-disulfonate), naphthoic (naphthoate), nicotinic (nicotinate), nitric (nitrate), octadecanonic (octadecanoate), oleic (oleate), orotic (orotate), oxalic (oxalate), pamoic (pamoate), pantothenic (pantothenate), phosphoric (phosphate), polygalacturonic (polygalacturonate), succinic (succinate), sulfosalicylic (sulfosalicylate), sulfuric (sulfate), tartaric (tartarate), p-toluenesulfonic (p-toluenesulfonate or tosylate) and xinafoic (xinafoate) acid, and the like. Such salts are sometimes referred to as acid addition salts.

The salts can be prepared by contacting one molar equivalent of ampreloxetine with about 0.95 to about 1.05 molar equivalents of acidic protons in the pharmaceutically acceptable acid. For example, one molar equivalent of ampreloxetine can be contacted with about one molar equivalent of hydrochloric acid to form ampreloxetine hydrochloride; or one molar equivalent of ampreloxetine can be contacted with about 0.5 molar equivalents of sulfuric acid to form an ampreloxetine sulfuric acid salt.

Such reactions are typically conducted in a diluent, such as dichloromethane, ethanol, ethyl acetate, isopropyl acetate, water and the like, at a temperature ranging from about −20° C. to about 65° C. for about 0.5 to about 12 hours or until the reaction is substantially complete. Upon completion of the reaction, the product is typically isolated using conventional procedures, such as filtration, chromatography, recrystallization, and the like. The product of such reactions may or may not be crystalline.

In some embodiments, a protected derivative of ampreploxetine, such as 4-[2-(2,4,6-trifluorophenoxymethyl)phenyl]piperidine-1-carboxylic acid tert-butyl ester, is contacted with a pharmaceutically acceptable acid, such as hydrochloric acid, to deprotect and form a pharmaceutically acceptable salt of ampreloxetine.

In some embodiments, the compound employed is ampreloxetine hydrochloride. In another embodiment, the compound is a crystalline hydrochloride salt of ampreloxetine characterized by a powder x-ray diffraction pattern comprising diffraction peaks at 2θ values of 4.44±0.2, 10.22±0.2, and 21.78±0.2. In another embodiment, the crystalline hydrochloride salt is further characterized by having one or more additional diffraction peaks at 2θ values selected from 8.11±0.2, 13.18±0.2, 16.06±0.2, 17.16±0.2, 18.38±0.2, 23.76±0.2, 26.32±0.2, 27.24±0.2, 29.60±0.2 and 31.94±0.2. In another embodiment, the compound is a crystalline hydrochloride salt of ampreloxetine characterized by a differential scanning calorimetry trace having a melting point of about 197±2° C.

In some embodiments, the crystalline hydrochloride salt of ampreloxetine contains from about 0 to about 2 weight percent water, e.g., the crystalline hydrochloride salt sorbs or desorbs water based on the relative humidity.

The crystalline hydrochloride salts of ampreloxetine employed can be prepared as described in the Examples herein or by the methods and procedures disclosed in U.S. Pat. Nos. 8,304,432; 8,304,433; and 8,247,433; and related patents.

### Pharmaceutical Compositions, Formulations and Dosage Forms

When used as disclosed herein, ampreloxetine or a pharmaceutically acceptable salt thereof is typically administered to a subject or patient in the form of a pharmaceutical composition or formulation. When describing compositions or formulations herein, ampreloxetine or a pharmaceutically acceptable salt thereof may be referred to as the “active agent” to distinguish it from other components of the formulation such as the carrier or excipient. Thus, the term “active agent” includes ampreloxetine as well as pharmaceutically acceptable salts thereof. Also, the terms “carrier” and “excipient” are used interchangeably herein and have the same meaning unless otherwise indicated.

Pharmaceutical compositions typically contain a therapeutically effective amount of the active agent. Those skilled in the art will recognize, however, that a pharmaceutical composition may contain more than a therapeutically effective amount, e.g., bulk compositions, or less than a therapeutically effective amount, e.g., individual unit doses designed for multiple administration to achieve a therapeutically effective amount.

Typically, a pharmaceutical composition will contain from about 0.01 to about 95 wt. % of active agent, including from about 0.01 to about 30 wt. %, such as from about 0.01 to about 10 wt. %, with the actual amount depending upon the formulation, the route of administration, the frequency of dosing, and so forth. For example, a pharmaceutical composition suitable as an oral dosage form may contain about 0.1 to about 10 wt. %, including from about 0.5 to about 5 wt. %, of active agent.

In one representative embodiment, the pharmaceutical composition contains from about 0.5 to about 20 mg of active agent per unit dose, including from about 1 to about 10 mg of active agent per unit dose. For example, the active agent may be formulated in 1, 2, 3, 4, 5, 6, 7, 8, 9, or 10 mg unit doses, such as 1 mg, 3 mg, 5 mg, 10 mg and 15 mg unit doses (where each amount refers to the free base equivalent amount of ampreloxetine). In a particular embodiment, the pharmaceutical composition contains about 10 mg of active agent per unit dose (free base equivalents).

Any conventional or suitable pharmaceutically acceptable carrier may be used in the pharmaceutical compositions. The choice of a particular carrier, or combinations of carriers, will depend on various factors, such as the mode of administration, dosage amount, frequency of dosing, timing of release of the active agent, and the like. In this regard, the preparation of a suitable pharmaceutical composition for a particular mode of administration is well within the scope of those skilled in the pharmaceutical arts, and carriers used in such compositions are commercially available. By way of further illustration, conventional formulations and formulation techniques are described in, e.g., Remington: The Science and Practice of Pharmacy, 23^{rd} Edition, Academic Press, Cambridge, M A (2020); and Loyd V. Allen, Jr. et al., Ansel's Pharmaceutical Dosage Forms and Drug Delivery Systems, 11^{th} Edition, Lippincott Williams & Wilkins, Philadelphia, PA (2017).

Representative examples of pharmaceutically acceptable carriers include, but are not limited to: sugars, such as lactose, glucose and sucrose; starches, such as corn starch and potato starch; cellulose, such as microcrystalline cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; compressed propellant gases, such as chlorofluorocarbons and hydrofluorocarbons; and other non-toxic compatible substances employed in pharmaceutical compositions.

Pharmaceutical compositions are typically prepared by thoroughly and intimately mixing or blending the active agent with a pharmaceutically acceptable carrier and any optional ingredients. The resulting uniformly blended mixture may then be shaped or loaded into tablets, capsules, pills, canisters, cartridges, vials, bottles, dispensers, and the like, using conventional procedures and equipment.

In some embodiments, the pharmaceutical composition is suitable for oral administration. Pharmaceutical compositions for oral administration may be in the form of, for example, capsules, tablets, pills, lozenges, cachets, dragees, powders, granules, solutions, suspensions, emulsions, elixirs, syrups, and the like; each containing a predetermined amount of the active agent.

When intended for oral administration in a solid dosage form (such as capsules, tablets, and the like), the pharmaceutical composition will typically comprise the active agent and one or more pharmaceutically acceptable solid carriers, such as sodium citrate or dicalcium phosphate. Solid dosage forms may also comprise: fillers or extenders, such as starches, microcrystalline cellulose, lactose, sucrose, glucose, mannitol, and/or silicic acid; binders, such as carboxymethylcellulose, alginates, gelatin, polyvinyl pyrrolidone, sucrose and/or acacia; humectants, such as glycerol; disintegrating agents, such as croscarmellose sodium, agar-agar, calcium carbonate, potato or tapioca starch, alginic acid, certain silicates, and/or sodium carbonate; solution retarding agents, such as paraffin; absorption accelerators, such as quaternary ammonium compounds; wetting agents, such as cetyl alcohol and/or glycerol monostearate; absorbents, such as kaolin and/or bentonite clay; lubricants, such as talc, calcium stearate, magnesium stearate, solid polyethylene glycols, sodium lauryl sulfate, and/or mixtures thereof; coloring agents; buffering agents; release agents; coating agents; sweetening, flavoring and/or perfuming agents; or preservatives and/or antioxidants.

Representative coating agents for tablets, capsules, pills, and the like include those used for enteric coatings, such as cellulose acetate phthalate, polyvinyl acetate phthalate, hydroxypropyl methylcellulose phthalate, methacrylic acid-methacrylic acid ester copolymers, cellulose acetate trimellitate, carboxymethyl ethyl cellulose, hydroxypropyl methyl cellulose acetate succinate, polyvinyl alcohol and the like.

Representative antioxidants include: water-soluble antioxidants, such as ascorbic acid, cysteine hydrochloride, sodium bisulfate, sodium metabisulfate, sodium sulfite, and the like; oil-soluble antioxidants, such as ascorbyl palmitate, butylated hydroxyanisole, butylated hydroxytoluene, lecithin, propyl gallate, alpha-tocopherol, and the like; and metal-chelating agents, such as citric acid, ethylenediamine tetraacetic acid, sorbitol, tartaric acid, phosphoric acid, and the like.

Pharmaceutical compositions may also be formulated to provide slow or controlled release of the active agent using, by way of example, hydroxypropyl methyl cellulose in varying proportions or other polymer matrices, liposomes and/or microspheres. In addition, the pharmaceutical composition may contain opacifying agents and may be formulated so that they release the active agent only, or preferentially, in a certain portion of the gastrointestinal tract, optionally, in a delayed manner. Examples of embedding compositions which can be used include polymeric substances and waxes. The active agent can also be in micro-encapsulated form, if appropriate, with one or more of the above-described excipients.

Suitable liquid dosage forms for oral administration include, by way of example, pharmaceutically acceptable emulsions, microemulsions, solutions, suspensions, syrups and elixirs. Liquid dosage forms typically comprise the active agent and an inert diluent, such as, for example, water, juice or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof. Suspensions may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In another embodiment, the pharmaceutical composition is suitable for topical administration, such as transdermal administration. For such administration, known transdermal delivery systems and excipients may be employed. For example, the active agent can be admixed with permeation enhancers, such as propylene glycol, polyethylene glycol monolaurate, azacycloalkan-2-ones, and the like, and incorporated into a patch or similar delivery system. Additional excipients, including gelling agents, emulsifiers and buffers, may be used in such transdermal compositions, if desired.

In another embodiment, the pharmaceutical composition is suitable for parenteral administration (e.g., by subcutaneous, intravenous, intramuscular, or intraperitoneal injection). For such administration, the active agent is provided in a sterile solution, suspension, or emulsion. Exemplary carriers for preparing such formulations include water, saline, low molecular weight alcohols such as propylene glycol, polyethylene glycol, oils, gelatin, fatty acid esters such as ethyl oleate, and the like. Parenteral formulations may also contain one or more solubilizers, stabilizers, preservatives, wetting agents, emulsifiers, and dispersing agents. These formulations may be rendered sterile by use of a sterile injectable medium, a sterilizing agent, filtration, irradiation, or heat.

A typical intravenous formulation is a sterile pH 4-7 aqueous solution comprising the active agent and a physiologically-acceptable aqueous carrier. Representative physiologically-acceptable aqueous carriers include, by way of example, Sterile Water for Injection, USP; Dextrose Injection, USP (e.g., 2.5, 5.0, 10, 20% dextrose, including 5% Dextrose Injection (D5/W)); Dextrose and Sodium Chloride Injection, USP (e.g., dextrose varying from 2.5 to 10% and sodium chloride varying from 0.12 (19 mEq sodium) to 0.9% (154 mEq sodium)); Mannitol Injection, USP, (e.g., 5, 10, 15, 20 and 25% mannitol); Ringer's Injection, USP (e.g., 147 mEq sodium, 4 mEq potassium, 4.5 mEq calcium and 156 mEq chloride per liter); Lactated Ringer's Injection, USP (e.g., 2.7 mEq calcium, 4 mEq potassium, 130 mEq sodium, and 28 mEq lactate per liter); Sodium Chloride Injection, USP (e.g., 0.9% sodium chloride) and the like. When administered to a subject, the active agent will typically be diluted in about 0.1 mL to about 10 mL of the aqueous carrier per mg of the active agent, such as about 0.5 to about 5 mL per mg. The dosing solution is then typically administered to the subject by intravenous infusion.

By way of illustration, representative pharmaceutical compositions can be prepared as described in the following examples.

### A. Hard Gelatin Capsules

The active agent (5 g), spray-dried lactose (485 g) and magnesium stearate (10 g) are thoroughly blended. The resulting composition is then loaded into hard gelatin capsules (500 mg of composition per capsule). Each capsule provides 5 mg of the active agent per unit dose suitable for oral administration.

### B. Hard Gelatin Capsules

The active agent (2 g) is thoroughly blended with starch (98 g), microcrystalline cellulose (98 g) and magnesium stearate (2 g). The mixture is then passed through a No. 45 mesh U.S. sieve and loaded into hard gelatin capsules (200 mg of composition per capsule). Each capsule provides 2 mg of the active agent per unit dose suitable for oral administration.

### C. Soft Gelatin Capsules

The active agent (5 g) is thoroughly blended with polyoxyethylene sorbitan monooleate (65 g) and starch powder (330 g). The mixture is then loaded into soft gelatin capsules (400 mg of composition per capsule). Each capsule provides 5 mg of the active agent per unit dose suitable for oral administration.

### D. Soft Gelatin Capsules

The active agent (1 g) is thoroughly blended with microcrystalline cellulose (290 g) and magnesium stearate (9 g). The mixture is then loaded into soft gelatin capsules (300 mg of composition per capsule). Each capsule provides 1 mg of the active agent per unit dose suitable for oral administration.

### E. Tablets

The active agent (10 g), starch (45 g) and microcrystalline cellulose (35 g) are passed through a No. 20 mesh U.S. sieve and mixed thoroughly. The resulting granules are dried at 50-60° C. and passed through a No. 16 mesh U.S. sieve. Separately, a solution of polyvinylpyrrolidone (4 g as a 10% solution in sterile water) is mixed with sodium carboxymethyl starch (4.5 g), magnesium stearate (0.5 g), and talc (1 g), and this mixture is passed through a No. 16 mesh U.S. sieve. The resulting mixture is then added to the granules. After mixing thoroughly, the mixture is compressed on a tablet press to form tablets weighing 100 mg each. Each tablet provides 10 mg of the active agent per unit dose suitable for oral administration.

### F. Tablets

The active agent (40 g) is thoroughly blended with microcrystalline cellulose (445 g), silicon dioxide fumed (10 g), and stearic acid (5 g). The mixture is then compressed on a tablet press to form tablets weighing 100 mg each. Each tablet provides 8 mg of the active agent per unit dose suitable for oral administration.

### G. Tablets

The active agent (10 g) is thoroughly blended with cornstarch (50 g), croscarmellose sodium (25 g), lactose (110 mg), and magnesium stearate (5 mg). The mixture is then compressed on a tablet press to form tablets weighting 200 mg each. Each tablet provides 10 mg of the active agent per unit dose suitable for oral administration.

### H. Tablets

The active agent (10 g) is thoroughly blended with cornstarch (230 g) and an aqueous solution of gelatin (50 g). The mixture is dried and ground to a fine powder. Microcrystalline cellulose (100 g) and magnesium stearate (10 g) are then admixed with the gelatin formulation, granulated and the resulting mixture compressed on a tablet press to form tablets weighing 200 mg each. Each tablet provides 5 mg of the active agent per unit dose suitable for oral administration.

### I. Syrup

The following ingredients are thoroughly mixed until all the solid ingredients are dissolved:

| Ingredients | Amount | |
|---|---|---|
| Active Agent | 0.5 | g |
| Citric acid | 2.1 | g |
| Artificial Raspberry Flavor | 2.0 | mL |
| Methyl Paraben | 2.0 | g |
| Propyl Paraben | 0.5 | g |
| Sorbitol Solution USP (64% | 1000.0 | mL |
| solution), to make | | |

The resulting syrup contains 5 mg of active agent per 10 mL of syrup suitable for oral administration.

### J. Sterile Intravenous Solution

The active agent (5 mg) is blended with 0.4 M sodium acetate buffer solution (2.0 mL). The pH of the resulting solution is adjusted to pH 4 using 0.5 N aqueous hydrochloric acid or 0.5 N aqueous sodium hydroxide, as necessary, and then sufficient water for injection is added to provide a total volume of 20 mL. The mixture is then filtered through a sterile filter (0.22 micron) to provide a sterile solution suitable for administration by intravenous infusion.

### Co-Administration and Combinations

If desired, ampreloxetine or a pharmaceutically acceptable salt thereof may be administered in combination with one or more other therapeutic agents (“secondary agents”).

Representative classes of therapeutic agents that can be administered in combination with ampreloxetine or a pharmaceutically acceptable salt thereof include, by way of example, α₁-adrenergic receptor (α₁-adrenoceptor) agonists, α₂-adrenergic receptor (α₂-adrenoceptor) antagonists, corticosteroids, norepinephrine precursors, cholinesterase inhibitors; or combinations thereof. Those skilled in the art will understand that the terms “α₁-adrenergic receptor agonist,” “α₂-adrenergic receptor antagonist,” “corticosteroid,” “norepinephrine precursor,” and “cholinesterase inhibitor” include all forms of compounds having the specified activity after administration to the subject, such as pharmaceutically acceptable salts, solvates, crystalline forms, polymorphs, prodrugs and the like. Similarly, the term “secondary agent” includes all forms of the secondary agent, such as pharmaceutically acceptable salts, solvates, crystalline forms, polymorphs, prodrugs and the like.

Representative examples of α₁-adrenergic receptor agonists include desglymidodrine, etilefrine, metaraminol, midodrine and the like, or, in each case, pharmaceutically acceptable salts thereof. Midodrine is a prodrug of desglymidodrine, which is an α₁-adrenergic receptor agonist. In some embodiments, the secondary agent is midodrine or a pharmaceutically acceptable salt thereof, such as midodrine hydrochloride.

Representative examples of α₂-adrenergic receptor antagonists include yohimbine and the like, or pharmaceutically acceptable salts thereof.

Representative examples of corticosteroids include fludrocortisone, fludrocortisone acetate and the like, or, in each case, pharmaceutically acceptable salts thereof. Fludrocortisone acetate is a prodrug of fludrocortisone. In some embodiments, the secondary agent is fludrocortisone acetate.

Representative examples of norepinephrine precursors include droxidopa or pharmaceutically acceptable salts thereof. In some embodiments, the secondary agent is droxidopa.

Representative examples of cholinesterase inhibitors include pyridostigmine or a pharmaceutically acceptable salt thereof. In some embodiments, the secondary agent is pyridostigmine or a pharmaceutically acceptable salt thereof, such as pyridostigmine bromide.

Ampreloxetine or a pharmaceutically acceptable salt thereof and the secondary agent may be either physically mixed to form a composition containing both agents; or each agent may be administered separately to the subject, either simultaneously or sequentially. For example, ampreloxetine or a pharmaceutically acceptable salt thereof can be combined with a secondary agent using conventional procedures and equipment to form a combination of agents comprising ampreloxetine or a pharmaceutically acceptable salt thereof and the secondary agent. Additionally, the agents may be combined with a pharmaceutically acceptable carrier to form a pharmaceutical composition comprising ampreloxetine or a pharmaceutically acceptable salt thereof, the secondary agent and a pharmaceutically acceptable carrier. In this embodiment, the components of the composition are typically mixed or blended to create a physical mixture. The physical mixture is then administered to the subject by any suitable route of administration, such as oral, topical or parenteral modes of administration.

Alternatively, the agents may remain separate and distinct before administration to the subject. In this embodiment, the agents are not physically mixed together before administration but are administered simultaneously or at separate times as separate compositions. Such compositions can be packaged separately or may be packaged together in a kit. When administered at separate times, the secondary agent will typically be administered less than 24 hours after administration of ampreloxetine or a pharmaceutically acceptable salt thereof, e.g., ranging anywhere from concurrent administration to about 24 hours post-dose. This is also referred to as sequential administration. Thus, for example, ampreloxetine or a pharmaceutically acceptable salt thereof can be orally administered simultaneously or sequentially with a secondary agent using two tablets (e.g., one tablet for each active agent), where sequentially includes being administered immediately before or after administration of ampreloxetine or a pharmaceutically acceptable salt thereof or at some other time (e.g., one hour before or after; or three hours before or after, etc.). Alternatively, the combination may be administered by different routes of administration, e.g., one orally and the other topically or parenterally.

When employed, the secondary agent is used in a therapeutically effective amount, i.e., in an amount that produces a therapeutically beneficial effect when co-administered with ampreloxetine or a pharmaceutically acceptable salt thereof. For example, such agents are typically employed in their approved dosage amounts. For example, midodrine hydrochloride is typically administered orally in an amount ranging from about 2.5 mg to about 10 mg up to three times per day; and droxidopa is typically administered orally in an amount ranging from about 100 mg to about 600 mg up to three times per day.

### Utility

Ampreloxetine or a pharmaceutically acceptable salts thereof (such as a hydrochloride salt) is being evaluated for the treatment of subjects with multiple system atrophy. Multiple system atrophy (MSA), also known as Shy-Drager syndrome, is a progressive neurodegenerative disorder characterized by a combination of symptoms that affect both the autonomic nervous system and movement. The initial symptoms of MSA are often difficult to distinguish from the initial symptoms of Parkinson's disease and include slowness of movement, tremor, or rigidity (stiffness); clumsiness or incoordination; impaired speech, a croaky, quivering voice; fainting or lightheadedness due to orthostatic hypotension; bladder control problems, such as a sudden urge to urinate or difficulty emptying the bladder. MSA is divided into two different types depending on the most prominent symptoms at the time an individual is evaluated: the parkinsonian type (MSA-P), with primary characteristics similar to Parkinson's disease (such as moving slowly, stiffness, and tremor) along with problems of balance, coordination, and autonomic nervous system dysfunction; and the cerebellar type (MSA-C), with primary symptoms featuring ataxia (problems with balance and coordination), difficulty swallowing, speech abnormalities or a quavering voice, and abnormal eye movements. The cause of MSA is unknown. A distinguishing feature of MSA is the accumulation of the protein alpha-synuclein in glia, the cells that support nerve cells in the brain. These deposits of alpha-synuclein particularly occur in oligodendroglia, a type of cell that makes myelin (a coating on nerve cells that lets them conduct electrical signals rapidly). A recent study indicates that a prion form of the alpha-synuclein protein may be the cause of the disease (Prusiner et al, PNAS, (2015) 112: E5308-17).

MSA is characterized by central autonomic pathway degeneration; however, peripheral postganglionic noradrenergic fibers and catecholamine reuptake mechanisms appear to be intact in MSA subjects thus maintaining sympathetic tone (Biaggioni, Pharmacological Reviews (2017) 69(1): 53-62). Normally, peripheral increases in norepinephrine concentration upon standing are counteracted by CNS sympatholytic activity mediated by norepinephrine-activated central α2-adrenoreceptors, thus buffering the peripheral pressor effect and maintaining postural normotension. In MSA, however, intact peripheral sympathetic postganglionic adrenergic fibers are essentially “disconnected” from CNS modulation thus allowing full unmasking of the norepinephrine pressor effect. Although residual sympathetic tone in these subjects cannot be modulated by baroreflex pathways or CNS input due to this “disconnection,” it can be targeted pharmacologically. Increasing peripheral sympathetic synaptic norepinephrine using ampreloxetine or a pharmaceutically acceptable salt thereof should induce a pressor effect in MSA subjects with nOH.

Ampreloxetine or a pharmaceutically acceptable salt thereof is typically administered to a subject in need of treatment in an amount ranging from about 0.5 mg to about 20 mg per day; or as needed. In some embodiments, the amount administered to the subject ranges from about 1 mg to about 10 mg per day. In some embodiments, the amount administered to the subject ranges from about 3 mg to about 10 mg per day. In some embodiments, the amount administered to the subject is 1, 2, 3, 4, 5, 6, 7, 8, 9, or 10 mg per day, including 1 mg, 3 mg, 5 mg, or 10 mg per day. In some embodiments, ampreloxetine or a pharmaceutically acceptable salt thereof is administered to the subject in an amount of about 10 mg per day. In some embodiments, ampreloxetine or a pharmaceutically acceptable salt thereof is administered to the subject in an amount of about 10 mg once per day. In some embodiments, ampreloxetine or a pharmaceutically acceptable salt thereof is administered to the subject in an amount of about 10 mg per day wherein the 10 mg amount is the free base equivalents of ampreloxetine. The amount administered to the subject, the route of administration and the frequency of administration will typically be determined by the physician treating the subject.

Ampreloxetine or a pharmaceutically acceptable salt thereof may be administered to a subject by any acceptable route of administration including, for example, oral, topical (including transdermal) and parenteral (including intravenous) modes of administration.

In some embodiments, ampreloxetine or a pharmaceutically acceptable salt thereof is administered to a subject orally in a solid or liquid dosage form. In a particular embodiment, the form administered to the subject is a solid dosage form including a tablet or capsule. In another particular embodiment, the form administered to the subject is a liquid dosage form including a solution, syrup, suspension or emulsion.

In another embodiment, the route of administration is topical. In a particular embodiment, the route of administration is transdermal using a transdermal patch.

In another embodiment, the route of administration is parenteral. In a particular embodiment, the route of administration is intravenous administration.

Ampreloxetine or a pharmaceutically acceptable salt thereof may be administered to the subject in a single daily dose (i.e., once a day); in multiple doses per day (e.g., twice, three times or four times daily); or in multiple doses per week (e.g., twice, three times, four times, five times or six times per week). Alternatively, a pharmaceutical composition may be administered continuously using, for examples, a transdermal patch. In a particular embodiment, ampreloxetine or a pharmaceutically acceptable salt thereof is administered to the subject once per day.

Unlike other therapies currently available, ampreloxetine may be administered for a prolonged time and show effectiveness for an extended period (e.g., more than two weeks). In some cases, depending on dosage regimen, ampreloxetine needs to be administered for a certain period to show effectiveness. For example, ampreloxetine or a pharmaceutically acceptable salt thereof is administered to a subject for at least about four weeks, at least about five weeks, at least about six weeks, at least about seven weeks, at least about eight weeks, at least about nine weeks, at least about ten weeks, at least about eleven weeks, at least about twelve weeks, at least about thirteen weeks, at least about fourteen weeks, at least about fifteen weeks, at least about sixteen weeks, at least about seventeen weeks, at least about eighteen weeks, at least about nineteen weeks, at least about twenty weeks, at least about twenty one weeks, at least about twenty two weeks, at least about twenty three weeks, at least about twenty four weeks, at least a year, or at least two years. In some embodiments, a pharmaceutical composition comprising a pharmaceutically acceptable carrier and about 10 mg (free base equivalents) of ampreloxetine or a pharmaceutically acceptable salt thereof, may be administered for aforementioned periods. Examples described herein shows some embodiments of such administration for extended period

In some embodiments, a pharmaceutical composition comprising a pharmaceutically acceptable carrier and about 10 mg (free base equivalents) of ampreloxetine or a pharmaceutically acceptable salt thereof, may be administered to a MSA patient or subject for at least about 8 weeks, to treat nOH. The administration may be made for at least about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks. In some embodiments, the administration may be made for more extended period, such as 6, 12, 18 and 24 months or more.

Such administration of ampreloxetine may result in improvement of nOH symptoms, which may be evidenced by reduction, from baseline, in one or more of: Orthostatic Hypotension Symptom Assessment (OHSA) items 1, 2, 3, 4, 5, 6, and composite score, Orthostatic Hypotension Daily Activities Scale (OHDAS) items 1, 2, 3, 4, and composite score, Orthostatic Hypotension Questionnaire (OHQ) composite score, and EQ-5D-5L Health Questionnaire scale score. In some embodiments, the reduction of OHSA (composite or any individual item), OHDAS (composite or any individual item), or OHQ composite score may be at least by 1, 2, 3, or 4 points. The administration of ampreloxetine may result in increase of seated or standing systolic blood pressure (e.g., greater than ˜20 mmHg), which may exceed the threshold for inducing syncope. In some embodiments, the administration may result in improvement in one or more of Patient Global Impression of Change (PGI-C), Patient Global Impression of Severity (PGI-S), Hospital Anxiety and Depression Scale (HADS) including Anxiety Total Score, Columbia Suicide Severity Rating Scale (C-SSRS), UMSARS Scale, COMPASS-31, Non-Motor Symptom Scale (NMSS), Burden Scale for Family Caregivers-short version (BSFC-s), incidence of falls, and time spent (e.g. 3 min or more) in standing position.

The patient or subject for the administration of ampreloxetine may be selected from different group of patients/subjects and may show effectiveness (as evidenced by aforementioned parameters) in wide variety of patients/subjects. For example, the patient or subject may have one or more of following properties: male or female; at least 30 years old; less than 65 years old, at least 65 years old. Before the treatment, the subject or patient may have Unified Multiple System Atrophy Rating Scale (UMSARS) Part IV less than 4; UMSARS Part IV of 4; UMSARS Part IV greater than 4; a sustained reduction in BP of ≥20 mmHg (systolic) or ≥10 mmHg (diastolic) within 3 min of being tilted up to ≥60° from a supine position as determined by a tilt-table test; at least 3, 4, 5, 6, or 7 on the OHSA item 1; a diagnosis of possible or probable MSA of Parkinsonian subtype (MSA-P) or cerebellar subtype (MSA-C); and/or plasma NE levels >100 pg/mL after being in seated position for 30 minutes. In some embodiments, the subject or patient may have had an onset of nOH less than about 0.5, 1, 1.1, 1.2, 1.3, 1.4, 1.5, 1.6, 1.7, 1.8, 1.9, 2, or 3 years before the treatment. In some embodiments, the subject or patient may have had an onset of nOH at least about 0.5, 1, 1.1, 1.2, 1.3, 1.4, 1.5, 1.6, 1.7, 1.8, 1.9, 2, or 3 years before the treatment. In some embodiment, the subject or patient may have received an MSA diagnosis less than about 0.5, 1, 1.1, 1.2, 1.3, 1.4, 1.5, 1.6, 1.7, 1.8, 1.9, 2, or 3 years before the treatment. In some embodiments, the subject or patient may have had MSA diagnosis at least about 0.5, 1, 1.1, 1.2, 1.3, 1.4, 1.5, 1.6, 1.7, 1.8, 1.9, 2, or 3 years before the treatment. In some embodiments, the subject or patient may have an OHSA composite score less than 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHSA composite score of at least 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHSA item 1, 2, 3, 4, 5, or 6 score less than 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHSA item 1, 2, 3, 4, 5, or 6 score of at least 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHDAS composite score less than 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHDAS composite score of at least 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHDAS item 1, 2, 3, or 4 score less than 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHDAS item 1, 2, 3, or 4 score of at least 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHQ composite score less than 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an OHQ composite score of at least 1, 1.5, 2, 2.5, 3, 3.5, 4, 4.3, 4.5, 4, 7, 5, 5.3, 5.5, 5.7, 6, 6.3, 6.5, 6.7, 7, 7.5, 8, 8.5, 9, 9.5 or 10 before the treatment. In some embodiments, the subject or patient may have an Orthostatic Hypotension Assessment Scale (OSHA) composite score of 5 or greater before the administration; and/or OSHA item 1 score of 7 or greater before the administration.

In some embodiments, the subject or patient may not have a systemic illness to produce autonomic neuropathy, including, but not limited to, amyloidosis and autoimmune neuropathies, and/or diabetes mellitus (DM). In some embodiments, the subject may not have a known intolerance to other NRIs or SNRIs. The subject or patient may not use concomitant antihypertensive medication for the treatment of essential hypertension. In some embodiments, the subject or patient may not use strong CYP1A2 inhibitors or inducers, midodrine, or droxidopa concurrently. In some embodiments, the subject or patient may not have known or suspected alcohol or substance abuse, clinically unstable coronary artery disease or had a major cardiovascular event (e.g., myocardial infarction) in the past 6 months, significant uncontrolled cardiac arrhythmia, history of complete heart block, significant QTc prolongation (≥450 msec for males and ≥470 msec for females), a history of untreated closed angle glaucoma, or treated closed angle glaucoma that, in the opinion of an ophthalmologist, a Montreal Cognitive Assessment (MoCA) <21, congestive heart failure (New York Heart Association [NYHA] Class 3 or 4), any malignant disease, other than carcinoma in situ of the cervix or basal cell carcinoma within the past 2 years prior to the treatment, a known gastrointestinal (GI) condition, psychiatric, neurological, or behavioral disorders that may interfere with the cognitive ability, a clinically significant abnormal laboratory finding(s) (e.g., alanine aminotransferase [ALT] or aspartate aminotransferase [AST] ≥3.0× upper limit of normal [ULN]; blood bilirubin [total] ≥3.0×ULN; estimated glomerular filtration rate (eGFR) <30 mL/min/1.73 m², or any abnormal laboratory value that could interfere with safety of the subject).

In some embodiments, the subject or patient who is responsive to, or treatable with ampreloxetine, may be identified first, before long-term administration (e.g. 8 weeks or more) of ampreloxetine, for more efficient and effective treatment. For example, the subject or patient may be administered with a pharmaceutical composition comprising a pharmaceutically acceptable carrier and about 10 mg of ampreloxetine for at least 8 weeks. If the subject or patient is responsive to ampreloxetine, the patient or subject may show improvement of nOH symptoms, which may be evidenced by reduction, from baseline, in one or more of: Orthostatic Hypotension Symptom Assessment (OHSA) items 1, 2, 3, 4, 5, 6, and composite score, Orthostatic Hypotension Daily Activities Scale (OHDAS) items 1, 2, 3, 4, and composite score, Orthostatic Hypotension Questionnaire (OHQ) composite score. In some embodiments, the reduction of OHSA (composite or any individual item), OHDAS (composite or any individual item), or OHQ composite score may be at least by 1, 2, 3, or 4 points. For example, administration for at least 8 weeks may result in at least 1 or 2 points reduction in the Orthostatic Hypotension Symptom Assessment (OHSA) Question 1 score (“dizziness, lightheadedness, feeling faint, or feeling like you might black out”). If the subject or patient is determined to be responsive to ampreloxetine, treatment may continue, and the pharmaceutical composition including ampreloxetine may be administered to the subject or patient for extended time (for at least 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 1 year, 2 years or 3 years). If the subject of patient is determined not to be responsive to ampreloxetine, the treatment may not continue and the pharmaceutical composition is no longer administered.

### EMBODIMENTS

Embodiment 1. A method for treating symptomatic neurogenic orthostatic hypotension in a subject having multiple system atrophy, the method comprising administering daily to the subject for at least about 22 weeks a pharmaceutical composition comprising a pharmaceutically acceptable carrier and about 10 mg (free base equivalents) of ampreloxetine or a pharmaceutically acceptable salt thereof, wherein:
(a) the subject being treated experiences symptoms of neurogenic orthostatic hypotension in the absence of treatment with the pharmaceutical composition as determined using the Orthostatic Hypotension Assessment Scale (OHSA); and
(b) administration of the pharmaceutical composition daily to the subject for at least about 22 weeks results in a measurable reduction in both (i) the subject's symptoms of dizziness, lightheadedness, feeling faint, or feeling like he or she might black out as determined using the subject's OHSA Question 1 score; and (ii) the subject's overall impression of symptom severity as determined using the subject's OHSA composite score.

Embodiment 2. The method of embodiment 1, wherein administration of the pharmaceutical composition daily for at least about 22 weeks further results in a measurable reduction in the subject's Orthostatic Hypotension Daily Activity Scale (OHDAS) composite score.

Embodiment 3. The method of embodiment 1 or 2, wherein administration of the pharmaceutical composition daily for at least about 22 weeks further results in a measurable increase in the subject's systolic blood pressure upon standing for 3 minutes.

Embodiment 4. The method of any one of embodiments 1 to 3, wherein the pharmaceutical composition is administered to the subject orally.

Embodiment 5. The method of any one of embodiments 1 to 4, wherein the pharmaceutical composition is administered to the subject once per day.

Embodiment 6. The method of any one of embodiments 1 to 5, wherein the subject has a supine plasma norepinephrine level before administration of the pharmaceutical composition of less than about 200 pg/mL.

Embodiment 7. The method of any one of embodiments 1 to 6, wherein the supine plasma norepinephrine level in the subject is less than about 350 pg/mL before treatment with the pharmaceutical composition

Embodiment 8. The method of any one of embodiments 1 to 7, wherein the supine plasma norepinephrine level in the subject is greater than about 500 pg/mL after about 8 weeks of treatment with the pharmaceutical composition.

Embodiment 9. The method of any one of embodiments 1 to 8, wherein ampreloxetine is administered as a hydrochloride salt.

Embodiment 10. The method of any one of embodiments 1 to 9, wherein ampreloxetine is administered as a crystalline hydrochloride salt characterized by a powder x-ray diffraction pattern comprising diffraction peaks at 2θ values of 4.44±0.2, 10.22±0.2, and 21.78±0.2.

Embodiment 11. The method of embodiment 10, wherein the crystalline hydrochloride salt ampreloxetine is further characterized by having one or more additional diffraction peaks at 2θ values selected from 8.11±0.2, 13.18±0.2, 16.06±0.2, 17.16±0.2, 18.38±0.2, 23.76±0.2, 26.32±0.2, 27.24±0.2, 29.60±0.2 and 31.94±0.2.

Embodiment 12. The method of any one of embodiments 1 to 11, wherein ampreloxetine is administered as a crystalline hydrochloride salt characterized by a differential scanning calorimetry trace having a melting point of about 197±2° C.

Embodiment 13. The method of any one of embodiments 1 to 12, wherein the pharmaceutically acceptable carrier comprises microcrystalline cellulose.

Embodiment 14. The method of any one of embodiments 1 to 12, wherein the pharmaceutically acceptable carrier comprises lactose.

Embodiment 15. The method of any one of embodiments 1 to 12, wherein the pharmaceutically acceptable carrier comprises magnesium stearate.

Embodiment 16. The method of any one of embodiments 1 to 12, wherein the pharmaceutically acceptable carrier comprises microcrystalline cellulose, lactose and magnesium stearate.

Embodiment 17. A method for reducing the magnitude of decline in quality of life of a subject having multiple system atrophy, the method comprising administering daily to the subject for at least about 22 weeks a pharmaceutical composition comprising a pharmaceutically acceptable carrier and about 10 mg (free base equivalents) of ampreloxetine or a pharmaceutically acceptable salt thereof, wherein the magnitude of decline in the quality of life of the subject is measured using the subject's self-rated health assessment on a vertical analogue scale.

Embodiment 18. The method of embodiment 17, wherein the vertical analogue scale comprises endpoints that are labeled “the best health you can imagine” and “the worst health you can imagine.”

Embodiment 19. The method of embodiment 17 or 18, wherein the vertical analogue scale is part of the EQ-5D-5L questionnaire.

Embodiment 20. The method of any one of embodiments 17 to 19, wherein the pharmaceutical composition is administered to the subject orally.

Embodiment 21. The method of any one of embodiments 17 to 20, wherein the pharmaceutical composition is administered to the subject once per day.

Embodiment 22. The method of any one of embodiments 17 to 21, wherein the magnitude of decline in the quality of life of the subject increases when the pharmaceutical composition is not administered to the subject.

Embodiment 23. The method of any one of embodiments 17 to 22, wherein the subject being treated experiences symptoms of neurogenic orthostatic hypotension in the absence of treatment with the pharmaceutical composition as determined using the Orthostatic Hypotension Assessment Scale (OHSA); and administration of the pharmaceutical composition daily to the subject for at least about 22 weeks results in a measurable reduction in the subject's symptoms of dizziness, lightheadedness, feeling faint, or feeling like he or she might black out as determined using the subject's OHSA Question 1 score.

Embodiment 24. The method of any one of embodiments 17 to 23, wherein the subject being treated experiences symptoms of neurogenic orthostatic hypotension in the absence of treatment with the pharmaceutical composition as determined using the Orthostatic Hypotension Assessment Scale (OHSA); and administration of the pharmaceutical composition daily to the subject for at least about 22 weeks results in a measurable reduction in the subject's overall impression of symptom severity as determined using the subject's OHSA composite score.

Embodiment 25. The method of any one of embodiments 17 to 24, wherein the subject being treated experiences symptoms of neurogenic orthostatic hypotension in the absence of treatment with the pharmaceutical composition as determined using the Orthostatic Hypotension Assessment Scale (OHSA); and administration of the pharmaceutical composition daily for at least about 22 weeks further results in a measurable reduction in the subject's Orthostatic Hypotension Daily Activity Scale (OHDAS) composite score.

Embodiment 26. The method of any one of embodiments 17 to 26, wherein administration of the pharmaceutical composition daily for at least about 22 weeks further results in a measurable increase in the subject's systolic blood pressure upon standing for 3 minutes.

Embodiment 27. The method of any one of embodiments 17 to 26, wherein the subject has a supine plasma norepinephrine level before administration of the pharmaceutical composition thereof of less than about 200 pg/mL.

Embodiment 28. The method of any one of embodiments 17 to 30, wherein the supine plasma norepinephrine level in the subject is less than about 350 pg/mL before treatment with the pharmaceutical composition.

Embodiment 29. The method of any one of embodiments 17 to 28, wherein the supine plasma norepinephrine level in the subject is greater than about 500 pg/mL after about 8 weeks of treatment with the pharmaceutical composition.

Embodiment 30. The method of any one of embodiments 17 to 29, wherein ampreloxetine is administered as a hydrochloride salt.

Embodiment 31. The method of any one of embodiments 17 to 30, wherein ampreloxetine is administered as a crystalline hydrochloride salt characterized by a powder x-ray diffraction pattern comprising diffraction peaks at 2θ values of 4.44±0.2, 10.22±0.2, and 21.78±0.2.

Embodiment 32. The method of embodiment 31, wherein the crystalline hydrochloride salt ampreloxetine is further characterized by having one or more additional diffraction peaks at 2θ values selected from 8.11±0.2, 13.18±0.2, 16.06±0.2, 17.16±0.2, 18.38±0.2, 23.76±0.2, 26.32±0.2, 27.24±0.2, 29.60±0.2 and 31.94±0.2.

Embodiment 33. The method of any one of embodiments 17 to 32, wherein ampreloxetine is administered as a crystalline hydrochloride salt characterized by a differential scanning calorimetry trace having a melting point of about 197±2° C.

Embodiment 34. The method of any one of embodiments 17 to 33, wherein the pharmaceutically acceptable carrier comprises microcrystalline cellulose.

Embodiment 35. The method of any one of embodiments 17 to 33, wherein the pharmaceutically acceptable carrier comprises lactose.

Embodiment 36. The method of any one of embodiments 17 to 33, wherein the pharmaceutically acceptable carrier comprises magnesium stearate.

Embodiment 37. The method of any one of embodiments 17 to 33, wherein the pharmaceutically acceptable carrier comprises microcrystalline cellulose, lactose and magnesium stearate.

Embodiment 38. A method for increasing the supine plasma norepinephrine level in a subject with multiple system atrophy, the method comprising administering daily to the subject for at least about 8 weeks a pharmaceutical composition comprising a pharmaceutically acceptable carrier and about 10 mg (free base equivalents) of ampreloxetine or a pharmaceutically acceptable salt thereof, wherein the supine plasma norepinephrine level in the subject is greater than about 500 pg/mL after about 8 weeks of treatment with the pharmaceutical composition.

Embodiment 39. The method of embodiment 38, wherein the pharmaceutical composition is administered to the subject orally.

Embodiment 40. The method of embodiment 37 or 39, wherein the pharmaceutical composition is administered to the subject once per day.

Embodiment 41. The method of any one of embodiments 37 to 40, wherein the subject has a supine plasma norepinephrine level before administration of the pharmaceutical composition of less than about 200 pg/mL.

Embodiment 42. The method of any one of embodiments 37 to 41, wherein the subject has a supine plasma norepinephrine level before administration of the pharmaceutical composition of less than about 350 pg/mL.

Embodiment 43. The method of any one of embodiments 37 or 42, wherein ampreloxetine is administered as a hydrochloride salt.

Embodiment 44. The method of any one of embodiments 37 to 43, wherein ampreloxetine is administered as a crystalline hydrochloride salt characterized by a powder x-ray diffraction pattern comprising diffraction peaks at 2θ values of 4.44±0.2, 10.22±0.2, and 21.78±0.2.

Embodiment 45. The method of embodiment 44, wherein the crystalline hydrochloride salt ampreloxetine is further characterized by having one or more additional diffraction peaks at 2θ values selected from 8.11±0.2, 13.18±0.2, 16.06±0.2, 17.16±0.2, 18.38±0.2, 23.76±0.2, 26.32±0.2, 27.24±0.2, 29.60±0.2 and 31.94±0.2.

Embodiment 46. The method of any one of embodiments 37 to 45, wherein ampreloxetine is administered as a crystalline hydrochloride salt characterized by a differential scanning calorimetry trace having a melting point of about 197±2° C.

Embodiment 47. The method of any one of embodiments 37 to 46, wherein the pharmaceutically acceptable carrier comprises microcrystalline cellulose.

Embodiment 48. The method of any one of embodiments 37 to 46, wherein the pharmaceutically acceptable carrier comprises lactose.

Embodiment 49. The method of any one of embodiments 37 to 46, wherein the pharmaceutically acceptable carrier comprises magnesium stearate.

Embodiment 50. The method of any one of embodiments 37 to 46, wherein the pharmaceutically acceptable carrier comprises microcrystalline cellulose, lactose and magnesium stearate.

### EXAMPLES

The following examples are provided to illustrate various aspects and embodiments of this disclosure and are not intended to limit the scope of this disclosure unless specifically indicated.

All reagents, starting materials and solvents used in the following examples were purchased from commercial suppliers (such as Sigma-Aldrich, St. Louis, MO and its affiliates) and were used without further purification unless otherwise indicated.

The following abbreviations have the following meanings unless otherwise indicated:
5-HT 5-hydroxytryptamine (or serotonin)
AE adverse event
bHCG beta human chorionic gonadotropin
BP blood pressure
BSA bovine serum albumin
BSFC-s Burden Scale for Family Caregivers-short version
CHO Chinese hamster ovary
COMPASS-31 Composite Autonomic Symptoms Score-31
C-SSRS Columbia Suicide Severity Rating Scale
DA dopamine
DAT dopamine transporter
DBP diastolic blood pressure
DHPG dihydroxyphenylglycol
DSM-IV-TR Diagnostic and Statistical Manual of Mental Disorders, Fourth Edition, Text Revision
ECG electrocardiogram
eGFR estimated glomerular filtration rate
ESC Enrollment Steering Committee
ET early termed
EQ-5D-5L EuroQol-5D-5L
FAS full analysis set
GDPR general data protection regulation
HADS Hospital Anxiety and Depression Scale
hDAT human dopamine transporter
HEPES 4-(2-hydroxyethyl)-1-piperazineethanesulfonic acid
Hgba1c hemoglobin A1c
HIPAA Health Insurance Portability and Accountability Act
hNET human norepinephrine transporter
hSERT human serotonin transporter
HR heart rate
MAO-I monoamine oxidase inhibitor
MAP mean arterial pressure
MAR missing at random
MMRM mixed model for repeated measures
MoCA Montreal Cognitive Assessment
MSA multiple system atrophy
MSA-C MSA of the cerebellar subtype
MSA-P MSA of the Parkinsonian subtype
NA noradrenaline
NE norepinephrine
NET norepinephrine transporter
NF National Formulary grade
NMSS Non-motor Symptom Scale
nOH neurogenic orthostatic hypotension
NRI norepinephrine reuptake inhibitor
NYHA New York Heart Association
OH orthostatic hypotension
OHQ Orthostatic Hypotension Questionnaire
OHDAS Orthostatic Hypotension Daily Activity Scale
OHSA Orthostatic Hypotension Symptom Assessment
OHSA #1 Orthostatic Hypotension Symptom Assessment Question 1
OL open label
PAF pure autonomic failure
PBO placebo
PD Parkinson's disease
PDQ-8 Parkinson's Disease Questionaire-8
PGI-C Patient Global Impression of Change
PGI-S Patient Global Impression of Severity
PI principal investigator
PK pharmacokinetic(s)
QD daily
RR respiratory rate
SAP statistical analysis plan
SBP systolic blood pressure
SERT serotonin reuptake transporter
symptomatic nOH symptomatic neurogenic orthostatic hypotension
SNRI serotonin norepinephrine reuptake inhibitor
Tris tris(hydroxymethyl)aminomethane
ULN upper limit of normal
UMSARS Unified Multiple System Atrophy Rating Scale
UPDRS Unified Parkinson's Disease Rating Scale
V1, V2, V3, etc. study visits

Other abbreviations used herein but not defined have their ordinary meaning as understood by persons having ordinary skill in the art to which they pertain.

### Example 1

### Radioligand Binding and Neurotransmitter Uptake Assays

The in vitro pharmacology of ampreloxetine at human recombinant and rat native monoamine transporters was characterized as described in Smith et al., Inter. J. Neuropsychopharmcol. (2015) 1-11; and Tsuruda et al., J. Pharmacol. Toxicol. Meth. (2010) 61:192-204. See also, e.g., U.S. Pat. Nos. 8,304,432 B2 and 8,304,433 B2. Radioligands were sourced commercially (Perkin Elmer LifeSciences or GE Healthcare Life Sciences).

Briefly, membranes prepared from HEK293 (Human Embryonic Kidney 293) or CHO-K1 (Chinese Hamster Ovary-K1) cells stably transfected with human recombinant SERT (HEK293-hSERT), NET (HEK293-hNET), or DAT (CHO-K1-hDAT) were incubated for 1 hr at 22° C. in the absence, or presence, of 4-[2-(2,4,6-trifluorophenoxymethyl)phenyl]piperidine and [³H]-citalopram (1.0 nM) for SERT, [³H]-nisoxetine (2.0 nM) for NET, and [³H]-WIN35428 (3.0 nM) for DAT in 50 mM Tris-HCl, 120 mM NaCl, 5 mM KCl, 0.025% BSA, 100 μM ascorbic acid, pH 7.4. Rat cortical membrane preparations were incubated with [³H]-citalopram (2.0 nM) for SERT or [³H]-nisoxetine (4.0 nM) for NET for 1 hr at 22° C. In neurotransmitter uptake assays, HEK293-hSERT, hNET, or hDAT cells, respectively, were pre-incubated for 30 min at 37° C. in the absence, or presence, of ampreloxetine in 7.5 mM HEPES, 12.5 mM Tris-HCl, 2.2 mM Na-phosphate, 120 mM NaCl, 5 mM KCl, 0.4 mM MgCl₂, 7.5 mM glucose, 1.7 mM CaCl₂, 250 μM ascorbic acid, 150 μM pargyline, 0.025% BSA, pH 7.4 prior to incubation with [³H]-5-HT (20 nM), [³H]-NE (40 nM), or [³H]-DA (100 nM) for 10 min. Rat cortical synaptosomes were incubated with [³H]-5-HT or [³H]-NE for 6 min and striatal synaptosomes with [³H]-DA for 6 min. Binding and uptake assays were terminated by rapid filtration and radioactivity determined by liquid scintillation spectroscopy. Final [³H]-neurotransmitter concentrations were significantly below the respective Kₘ such that pIC₅₀ approximated functional pKᵢ. Selectivity for NET (rounded to one significant figure) was determined as follows:

Selectivity=10^{(pKi or pIC50 at NET minus pKi or pIC50 at SERT or DAT)}.

The in vitro pharmacological profile of ampreloxetine was similar at human and rodent monoamine transporters as shown in Table 1.

TABLE 1
In Vitro Pharmacology at Human Recombinant and
Rat Native SERT, NET, and DAT Transporters

| | | | |
|---|---|---|---|
| Species | SERT | NET | DAT |

Neurotransmitter Uptake Inhibition: pIC₅₀

| | | | | |
|---|---|---|---|---|
| Human | 8.0 (7.8, 8.2) | 8.6 (8.4, 8.7) | 6.8 | (6.6, 6.9) |
| Rat | 7.9 (7.8, 7.9) | 8.9 (8.6, 9.1) | 6.9 | (6.8, 6.9) |

Transporter Binding: pKᵢ

| | | | | |
|---|---|---|---|---|
| Human | 8.5 (8.5, 8.6) | 8.8 (8.8, 8.9) | 6.7 | (6.7, 6.8) |

| | | | |
|---|---|---|---|
| Rat | 8.5 (8.3, 8.6) | 8.7 (8.5, 8.9) | N.D. |

Data are expressed as mean pIC₅₀ (negative decadic logarithm IC₅₀) and pKᵢ (negative decadic logarithm Kᵢ) values.
Data represents mean (with 95% confidence intervals in parentheses) from 3 to 9 individual experiments.
N.D. = not determined.

The data in Table 2 demonstrate that ampreloxetine is a potent inhibitor of NET and SERT, but not DAT, with 4-fold higher potency for inhibition of NET over SERT. Similarly, ampreloxetine is a potent inhibitor of both [³H]-NE and [³H]-5-HT uptake into rat cortical synaptosomes, with an apparent functional selectivity (10-fold) for NET over SERT, similar to that observed at human transporters. Consistent with the functional inhibition studies, ampreloxetine exhibited a high affinity for binding to human NET and SERT, but not DAT (Table 2). Apparent binding affinity values for rat-native NET and SERT in membranes prepared from rat cortices were similar (overlapping confidence intervals) to the corresponding values at human transporters, consistent with a lack of species dependence (Table 2).

### Example 2

### Ex Vivo Transporter Occupancy Studies

Adult male Sprague Dawley rats (Charles River) were housed under controlled laboratory conditions (temperature at 21±1° C.) on a 12:12 hour light-dark cycle. Animals were given free access to food and water upon arrival to the facility and animals were acclimatized to their holding room for at least 48 hours. Animals were fasted but allowed free access to water for 15-18 hours prior to dosing.

Rats (n=6/timepoint/dose level) received a single oral dose of 4-[2-(2,4,6-trifluorophenoxymethyl)phenyl]piperidine (0.3, 1, 5, 10, 30, and 60 mg/kg) and were euthanized by decapitation at specified time points (0.5, 2, 4, 6, and 8 hr for 5 mg/kg dose level; 2 hr for 0.3, 1, 10, 30, and 60 mg/kg dose levels) post-administration. Spinal cords were dissected for ex vivo transporter occupancy and PK assessments from the same animals. The spinal cord was harvested by hydraulic extrusion using phosphate-buffered saline, and the lumbar segment dissected and frozen on dry ice. The remaining spinal cord segments were collected and homogenized in water (25% w/w) for PK analysis. All samples were stored at −80° C. until analysis.

A kinetic radioligand binding assay was used to determine NET and SERT occupancy in rat spinal cord, as described previously in Bourdet et al., J. Pharm. Exp. Ther. (2012) 341:137-145. PK/PD parameters were estimated by a compartmental modeling approach (WinNonlin Version 5.0.1, Pharsight Corporation). One- and two-compartment PK models with first-order absorption and elimination were evaluated. The one-compartment model was selected. The pharmacodynamics model was an effect compartment Eₘₐₓ model linked directly to the central PK compartment (WinNonlin PK Model 3, PD Model 101). Selection of models was based upon best fit in terms of visual inspection, Akaike Information Criteria, and weighted residual sum of squares using the Gauss-Newton minimization method. The following parameters were estimated:
k01 (hr⁻¹): First-order absorption rate constant.
V/F (L/kg): Volume of the central compartment divided by oral bioavailability
k10 (hr⁻¹): Elimination rate constant from the central compartment
Eₘₐₓ (% occupancy): Maximal SERT or NET occupancy in spinal cord
EC₅₀ (ng/mL): Plasma 4-[2-(2,4,6-trifluorophenoxymethyl)phenyl]piperidine concentration associated with 50% SERT or NET occupancy
kₑₒ (hr⁻¹): First-order equilibration rate constant between the central pharmacokinetic compartment and the pharmacodynamic effect compartment

PK and PD parameter estimates derived from the effect compartment PK/PD analysis for NET and SERT occupancy are shown in Table 2.

TABLE 2
Pharmacokinetic and Pharmacodynamic Parameter Estimates
for Ampreloxetine Norepinephrine and Serotonin
Transporter Occupancy in Rat Spinal Cord

| | | |
|---|---|---|
| Parameter | SERT | NET |

| | | | | |
|---|---|---|---|---|
| Eₘₐₓ (% occupancy) | 79.0 | (53) | 92.0 | (19) |
| EC₅₀ (ng/mL) | 50.8 | (87) | 11.7 | (6.8) |
| kₑₒ (hr⁻¹) | 11.0 | (86) | 1.78 | (57) |
| k01 (hr⁻¹) | 0.777 | (108) | | |
| k10 (hr⁻¹) | 0.319 | (81) | | |
| V/F (L/kg) | 54.8 | (66) | | |

*Final parameter estimates are listed with the coefficient of variation (% CV) provided in parentheses.

As shown in Table 2, the estimated EC₅₀ for occupancy was 11.7 ng/mL for NET and 50.8 ng/mL for SERT in rat spinal cords. Accounting for species differences in plasma protein binding (90.2% and 79.1% in rat and human, respectively), the projected human plasma EC₅₀ values were 5.5 ng/mL for NET and 23.9 ng/mL for SERT.

### Example 3

### Cardiovascular Model in an Anesthetized Rat

These studies were conducted to assess the effect of a single administration of ampreloxetine on heart rate (HR) and mean arterial pressure (MAP) in anesthetized rats. Using this cardiovascular model, the intrinsic effect on HR and the inhibition of tyramine pressor response were evaluated as surrogate measures that reflect the ability of ampreloxetine to inhibit norepinephrine transporters in the periphery.

### A. Experimental Design

Normotensive male Sprague-Dawley rats weighing between 250-350 g were anesthetized with an intraperitoneal injection (IP) of thiobutabarbital (Inactin). All animals were kept under complete anesthesia (i.e., absence of response to toe pinch test) for the surgery and for the duration of the study. The right common carotid artery and jugular vein were isolated and catheterized. The trachea was intubated to keep the airway open during the study. After completion of surgery, the arterial catheter was connected to a pressure transducer and baseline blood pressure [Systolic (SBP), Mean Arterial (MAP) and Diastolic (DBP)] and heart rate (HR) were recorded using the Notocord-HEM data acquisition system. Following at least 60 min of baseline (i.e., the last 10 min was stable), vehicle (10% Tween20, 2 mL/kg, IP) was administered and any potential effect was monitored for at least 10 min. After this period, rats were injected with either vehicle or ampreloxetine (0.01-30 mg/mL, 2 mL/kg, IP) and changes in MAP and HR were monitored for 25 min. Rats were then challenged intravenously via the jugular vein catheter, with non-cumulative bolus doses of tyramine (0.03, 0.1, 0.3, and 1 mg/kg, 1 mL/kg, IV) given at 5 min intervals. After the last dose of tyramine, data acquisition continued for another 10 min before the experiment was terminated. In a separate group of animals, blood was collected to assess the concentration of ampreloxetine in plasma at 15 min and 60 min after dosing. Free plasma concentrations obtained from 15 min were used to construct the concentration response curve (CRC) to MAP and HR while concentrations from 60 min were used for the tyramine CRC. Animals were euthanized by carbon dioxide asphyxiation followed by thoracotomy.

### B. Data Analysis

Intrinsic hemodynamic effects were reported as the maximum change in MAP or HR induced by ampreloxetine before tyramine challenge. Inhibition of tyramine effect was normalized to the response to 1 mg/kg dose of tyramine in the vehicle control group. Concentration response curves (CRCs) of the change in MAP, HR and inhibition of tyramine response were analyzed through iterative curve fitting to a logistic equation using Prism 5.00™ (GraphPad, Inc.). The equation used was as follows:

Y=(Bottom+Top−Bottom)/(1+10\hat{ }((Log EC50−X)*HillSlope))

where X is the logarithm of dose, Y is the response, Y starts at Bottom (constrained to 0 for all) and goes to Top with a sigmoid shape. When change in MAP or HR was reduced at the higher dose(s), a more accurate estimate of Top (i.e., maximum efficacy) was obtained by carrying over the maximum effect which occurred at a lower dose to all the subsequent higher doses. For the tyramine CRC, the TOP was constrained to 100. Potency of the pressor effect was reported as MAP PC₁₀ which is the free plasma concentration that produced a change in MAP of 10 mm Hg and the potency of the HR effect was reported as HR PC₂₅ which is the free plasma concentration that produced a change in HR of 25 bpm. Lastly, the potency to inhibit the tyramine effect was reported as Tyr EC₅₀ which is the concentration that produced 50% inhibition of tyramine-induced (1 mg/kg, IV) increase in SBP.

Inhibitory potencies (pIC₅₀ values) obtained from measuring the inhibition of labelled 5-HT uptake by rat serotonin transporters (rSERT) in rat cortical synaptosomes were converted to IC₅₀ values using this formula:

Y=(10\hat{ }(−x))x(10\hat{ }9)

### C. Results

Ampreloxetine (0.01-30 mg/kg, IP) dose dependently increased MAP and HR in anesthetized rats. When plotted against the corresponding free plasma concentration for each dose, the estimated MAP and HR potencies were 101.4 nM (MAP EC₁₀) and 8.6 nM (HR EC₂₅). The maximum changes in MAP and HR were 13.2 (4.4-22.1) mm Hg and 28.1 (22.4-33.7) bpm, respectively. Ampreloxetine also inhibited tyramine-induced increases in SBP. When expressed as % inhibition of response to 1 mg/kg of intravenously administered tyramine, the estimated potency of ampreloxetine was 0.86 nM.

### D. Conclusion

In a cardiovascular model in anesthetized rat, ampreloxetine exhibited potent inhibition of tyramine pressor response and tachycardia which is consistent with inhibition of NET in the periphery.

### Example 4

### Preparation of Oral Dosing Solutions

Oral dosing solutions of ampreloxetine hydrochloride were prepared in two steps. First, a 3 mg/mL aqueous stock solution was prepared and then, oral solutions in filtered apple juice having different dose strengths were prepared prior to dosing.

### A. Preparation of Stock Solution

Ampreloxetine hydrochloride (500 mg, 89.9% purity) was added to a 250 mL clear glass bottle. Sterile Water for Injection (150 mL) was added and the bottle was capped. The bottle was gently swirled in a circular motion until no solid material was observed (about 20 minutes). If needed, the bottle can also be sonicated. The bottle was labeled and the stock solution (3 mg/mL) was used within 2 hours or stored in a refrigerator at 2-8° C. until use. Any stock solution not used within 6 days of initial preparation was discarded.

### B. Preparation of Oral Dosing Solutions

Oral dosing solutions having seven different dose strengths were prepared for use in either Part A or Part B of the clinical studies. The dose strengths prepared and the amounts used to prepare each dose strength were as shown in Tables 3 and 4:

TABLE 3
Oral Dosing Solutions (Part A)

| Dose | Final Conc. | Stock Vol. | Apple Juice | Oral Solution | Final Dosing |
|---|---|---|---|---|---|
| (mg) | (μg/mL) | (mL) | Vol. (mL) | Prepared (mL) | Vol. (mL) |
| 1 | 100 | 1 | 29 | 30 | 10 |
| 2.5 | 250 | 2 | 22 | 24 | 10 |
| 5 | 500 | 4 | 20 | 24 | 10 |
| 10 | 1000 | 10 | 20 | 30 | 10 |

Oral Dosing Solutions (Part B)

| Dose | Final Conc. | Stock Vol. | Apple Juice | Oral Solution | Final Dosing |
|---|---|---|---|---|---|
| (mg) | (μg/mL) | (mL) | Vol. (mL) | Prepared (mL) | Vol. (mL) |
| 1 | 100 | 1 | 29 | 30 | 10 |
| 3 | 300 | 3 | 27 | 30 | 10 |
| 7 | 700 | 7 | 23 | 30 | 10 |
| 15 | 1500 | 15 | 15 | 30 | 10 |

To prepare the oral dosing solutions, the stock solution (3 mg/mL) was removed from the refrigerator and visually checked for any precipitation. If precipitation was present, the stock solution was re-prepared.

Apple juice (at least 40 mL, Mott's 100% Original Apple Juice) was drawn into a 50 mL syringe and a syringe filter (25 mm PVDF Syringe Filter, 0.2 μm, Pall Life Sciences) was attached to the syringe. The apple juice was filtered through the syringe filter with the first 3 mL being discarded and the remaining apple juice being collected in a 125 mL amber bottle.

The amount of stock solution (3 mg/mL) shown in Table 3 or 4 (Stock Volume) was then added to a new 125 mL amber bottle and the corresponding amount of filtered apple juice shown in Table 3 or 4 (Apple Juice Volume) was added to the bottle. The bottle was capped and the contents were mixed by swirling in a circular motion for more than 2 minutes. The resulting solution was stored at ambient room temperature for up to 18 hours before dosing the subject. Prior to dosing the subject, a 10 mL aliquot of the oral solution was transferred into a new 125 mL amber bottle.

### Example 5

### Preparation of Oral Dosing Tablets for Phase 3 Study

Microcrystalline cellulose (35.32 kg; AVICEL Microcrystalline Cellulose, NF, Ph. Eur Type PH-112) and anhydrous lactose (21.00 kg; anhydrous 60M, NF, EP) were loaded into a blending bin and blended for 5 minutes at 20 rpm. The resulting mixture was transferred into a double polyethylene (PE)-lined container (“Premix 1”) and separated into two parts, one part weighing about 10 kg (“Premix 1A) and the other part comprising the remainder (“Premix 1B”). About one-half of Premix 1A was added to a bin followed by ampreloxetine monohydrochloride (3.382 kg; milled), and then the second half of Premix 1A was added. The resulting mixture was blended for 10 minutes at 20 rpm and then transferred into a double PE-lined container (“Active Premix 2”). About one-half of Premix 1B was passed through a mill fitted with a 1.0 mm screen at 1000 rpm (800-1200 rpm), impact forward, followed by Active Premix 2 and then the second half of Premix 1B. The milled materials were collected into a double PE-lined container (“Milled Premix 3”). Milled Premix 3 was passed through a 40-mesh sieve, and the sifted material was transferred into a blending bin and blended for 25 minutes at 20 rpm (“Premix 4”). Two full scoops of Premix 4 were removed and mixed manually with magnesium stearate (300.0 g, NF) in a polyethylene bag. This mixture was then passed through a 40-mesh hand screen. The screened mixture was then added back into the blending bin and the entire mixture was blended for 5 minutes at 20 rpm (“Final Blend’). The Final Blend was compressed into 200 mg tablets using a tablet press and the tablets were passed through a deduster and metal detector. The acceptable tablets were collected into double PE-lined containers.

Purified water (12.48 kg, USP) was added into a clean stainless steel solution preparation vessel and the mixer speed was adjusted to form a vortex. A polyvinyl alcohol-based film coating (3.12 kg; OPADRY II White 85G18490, Colorcon, Inc., West Point, PA) was added into the vortex in the mixing tank and the mixing speed was reduced so there was no longer a vortex and mixing was continued for at least 45 minutes or until the color was uniformly dispersed by visual observation. Gentle mixing was maintained before and during the coating process. The tablets prepared above were loaded into a coating pan and the pan speed was set at 6 rpm. The coating solution was pumped into the coating pan. The coating solution weight change was monitored as it was pumped into the coating pan and addition of the coating solution was stopped when a 4% tablet dried weight gain was achieved based on coating suspension usage. The resulting white tablets contained about 10 mg of ampreloxetine (free base equivalents).

### Example 6

A Phase 3 4-Week Clinical Study in Subjects with Symptomatic nOH

This study was a Phase 3, randomized, double-blind, placebo-controlled, parallel-group, multicenter study to evaluate efficacy, safety, and tolerability of ampreloxetine (administered as ampreloxetine hydrochloride) in subjects with primary autonomic failures (MSA, PD, or PAF) and symptomatic nOH after four weeks of treatment. Subjects participated in the study for approximately 10 weeks.

A total of 185 subjects completed this study. Subjects were randomized in a 1:1 ratio to each of either ampreloxetine or placebo group stratified by the subject's disease type (MSA, PD, or PAF). 98 subjects were randomized to the ampreloxetine group (8 subjects withdrew during the study) and 97 subjects were randomized to the placebo group (2 subjects withdrew during the study). 37% of subjects enrolled in the study had MSA.

### A. Study Population

This study enrolled adult subjects with confirmed symptomatic nOH due to MSA, PD, or PAF, who met all of the inclusion criteria and none of the exclusion criteria defined in the study protocol.

1. Inclusion Criteria

Subjects who met the following criteria were eligible for study enrollment.

(a) Subject was male or female and at least 30 years old.

(b) For female subjects only: Subject was nonpregnant and nonlactating. A woman of childbearing potential must have a documented negative pregnancy test at screening. A woman was considered to be of childbearing potential unless she was postmenopausal (amenorrheic for at least 2 years) or documented to be surgically sterile (bilateral tubal ligation or total hysterectomy). A female subject was admitted to the study on the basis of a negative urine pregnancy test. If the urine bHCG (beta human chorionic gonadotropin) test was positive, a serum bHCG test was performed. The pregnancy test must be confirmed negative for a subject to be eligible for this study.

(c) During the study and for 30 days after receiving the last dose of the study drug, females of childbearing potential or males capable of fathering children agreed to use highly effective birth control measures (failure rate <1% when used consistently and correctly) or agreed to abstain from sexual intercourse.

(d) Subject met the diagnostic criteria of nOH, as demonstrated by a sustained reduction in BP of ≥20 mmHg (systolic) or ≥10 mmHg (diastolic) within 3 min of being tilted up to ≥60° from a supine position as determined by a tilt-table test.

(e) Subject scored at least a 4 on the Orthostatic Hypotension Symptom Assessment Question #1 at randomization visit.

(f) For subjects with PD only: subject had a diagnosis of PD according to the United Kingdom Parkinson's Disease Society (UKPDS) Brain Bank Criteria (1992).

(g) For subjects with MSA only: subject had a diagnosis of possible or probable MSA of the Parkinsonian subtype (MSA-P) or cerebellar subtype (MSA-C) according to The Gilman Criteria (2008).

(h) For subjects with PAF only: subject had documented impaired autonomic reflexes, including the Valsalva maneuver performed within 24 months from the date of randomization.

(i) Subject had plasma NE levels >100 pg/mL after being in seated position for 30 minutes.

(j) Subject was willing and able to provide signed and dated written informed consent to participate prior to initiation of any study related procedures.

(k) Subject was able to communicate well with the Investigator and clinic staff, understood the expectations of the study and was able to comply with the study procedures, requirements, and restrictions.

2. Exclusion Criteria

Subjects who met any of the following criteria were not eligible for study enrollment.

(a) Subject had a known systemic illness known to produce autonomic neuropathy, including but not limited to amyloidosis and autoimmune neuropathies. Subject had diabetes mellitus and diagnosis of PAF. Subject with diabetes mellitus and either MSA or PD were evaluated on a case-by-case basis by the medical monitor and considered ineligible unless they meet all of the following criteria:
i. Well controlled type-2 DM in treatment with only oral medications and diet;
ii. HgbA1C of ≤7.5% performed during screening or up to 12 weeks before screening;
iii. No clinically evident peripheral neuropathy (e.g., normal sensory examination on peripheral extremities);
iv. No known retinopathy (e.g., annual ophthalmic exam was sufficient); and
v. No nephropathy (e.g., absence of albuminuria and GFR >60).

(b) Subject had a known intolerance to other NRIs or serotonin norepinephrine reuptake inhibitors (SNRIs).

(c) Subject used concomitant antihypertensive medication for the treatment of essential hypertension.

(d) Subject had used strong CYP1A2 inhibitors or inducers within 7 days or 5 half-lives, whichever was longer, prior to randomization or requires concomitant use until the follow-up visit.

(e) Subject had changed dose, frequency, or type of prescribed medication for orthostatic hypotension within 7 days prior to randomization visit (midodrine and droxidopa (if applicable) must be tapered off at least 7 days prior to randomization).

(f) Subject had known or suspected alcohol or substance abuse within the past 12 months (DSM-IV-TR® definition of alcohol or substance abuse).

(g) Subject had a clinically unstable coronary artery disease, or major cardiovascular or neurological event in the past 6 months.

(h) Subject had used any monoamine oxidase inhibitor (MAO-I) within 14 days prior to randomization.

(i) Subject had a history of untreated closed angle glaucoma, or treated closed angle glaucoma that, in the opinion of an ophthalmologist, might result in an increased risk to the subject.

(j) Subject had any significant uncontrolled cardiac arrhythmia.

(k) Subject had a Montreal Cognitive Assessment (MoCA) ≤23.

(l) Subject was unable or unwilling to complete all protocol specified procedures including questionnaires.

(m) Subject had a myocardial infarction in the past 6 months or has current unstable angina.

(n) Subject had known congestive heart failure (New York Heart Association [NYHA] Class 3 or 4).

(o) Subject had any malignant disease other than carcinoma in situ of the cervix or basal cell carcinoma within the past 2 years prior to screening.

(p) Subject had a known gastrointestinal (GI) condition, which in the Investigator's judgment, may affect the absorption of study medication (e.g., ulcerative colitis, gastric bypass).

(q) Subject has psychiatric, neurological, or behavioral disorders that may interfere with the ability of the subject to give informed consent or interfere with the conduct of the study.

(r) Subject was currently receiving any investigational drug or had received an investigational drug within 30 days of dosing. An investigational drug was defined as nonregulatory agency approved drug (e.g., Food and Drug Administration).

(s) Subject had a clinically significant abnormal laboratory finding(s) (e.g., alanine aminotransferase [ALT] or aspartate aminotransferase [AST] >3.0× upper limit of normal [ULN]; blood bilirubin [total] >1.5×ULN; estimated glomerular filtration rate (eGFR) <30 mL/min/1.73 m², or any abnormal laboratory value that could interfere with safety of the subject).

(t) Subject had demonstrated a history of lifetime suicidal ideation and/or suicidal behavior, as outlined by the C-SSRS (Baseline/Screening Version) subject should be assessed by the rater for risk of suicide and the subject's appropriateness for inclusion in the study.

(u) Subject had a concurrent disease or condition that, in the opinion of the Investigator, would confound or interfere with study participation or evaluation of safety, tolerability, or pharmacokinetics of the study drug.

(v) Subject had known hypersensitivity to ampreloxetine (ampreloxetine hydrochloride), or any excipients in the formulation.

(w) Subject had (i) confirmed severe acute respiratory syndrome coronavirus 2 (SARS-CoV-2) documented with coronavirus disease 2019 [COVID-19] positive test result, OR (ii) was suspected of SARS-CoV-2 infection (clinical features without documented test results two weeks after resolution of symptoms and remains asymptomatic until Day 1), OR (iii) had been in close contact with a person with known (or suspected) SARS-CoV-2 infection and remains asymptomatic until Day 1.

3. Prohibitions and Restrictions

The following were prohibited or restricted during study participation as specified.

(a) Subjects were required to stop the concomitant use of strong CYP1A2 inhibitors and inducers 7 days or 5 half-lives, whichever is longer, prior to randomization. This restriction applied to concomitant medications, herbal supplements (e.g., St John's Wort), and ordinary dietary intake.

(b) Prescribed medications for OH other than fludrocortisone were prohibited.

(c) Alpha blockers were prohibited (e.g., Prazosin, Terazosin, Doxazosin, Silodosin, Alfuzosin, Tamsulosin).

(d) Norepinephrine reuptake inhibitors (NRIs) (e.g., atomoxetine and reboxetine) and serotonin and norepinephrine reuptake inhibitors (SNRIs) (e.g., duloxetine, milnacipran, levomilnacipran, venlafaxine, desvenlafaxine) were prohibited.

(e) Psychostimulants (e.g., amphetamine, dextroamphetamine, methylphenidate, pemoline) were prohibited.

(f) Subjects were requested to refrain from making any significant dietary changes throughout the duration of the study. Subjects were reminded to maintain an adequate fluid intake during their scheduled visits.

### B. Test Product, Dose, and Route of Administration; Regimen: Duration of Treatment

Subjects were randomized in a 1:1 ratio to receive either ampreloxetine (test product) or placebo (reference product) once daily through the end of the treatment period.

The test product was ampreloxetine supplied as a 10-mg tablet in 35-count high-density polyethylene bottles labeled in a blinded fashion (e.g., bottle label for test product was indistinguishable from reference product except for a coded, unique bottle number).

Ampreloxetine was administered orally without regard to food at approximately the same time each morning and taken with approximately 8 ounces of water.

Subjects randomized to receive ampreloxetine began taking study medication on Day 2 in the morning at a dose of 10 mg and continued to take this dose once daily through the end of the treatment period.

### D. Reference Product, Dose, and Route of Administration: Regimen: Duration of Treatment

The reference product was placebo tablets that were supplied to match the test product in excipient content (except for ampreloxetine), appearance, tablet count, and in packaging (e.g., bottle label for reference product was indistinguishable from test product except for a coded, unique bottle number).

Placebo was administered orally without regard to food at approximately the same time each morning and taken with approximately 8 ounces of water.

Subjects randomized to receive placebo began taking study medication on Day 2 in the morning and continued to take placebo once daily through the end of the treatment period.

### E. Study Execution

For purposes of this study, symptomatic neurogenic orthostatic hypotension was defined as:
(a) A sustained reduction of BP of ≥20 mmHg (systolic) or ≥10 mmHg (diastolic) within 3 minutes of standing or tilted-up to ≥60° elevation from a supine position; and
(b) A score of at least a 4 on the Orthostatic Hypotension Symptom Assessment Question #1.

The study consisted of three periods: (i) 4-week screening, (ii) 4-week randomized treatment, and (iii) 2-week follow up.

After signing the informed consent, the subject entered a screening period of up to 4 weeks to confirm eligibility. At the Screening visit, which was performed in the clinic for all subjects, the subject provided a comprehensive medical history of their disease and treatments. The subject's disease was characterized and documented by the Investigator. The subject received an assessment of their physical condition, including safety and laboratory evaluations. The presence of symptomatic nOH symptoms and the reported sensation of dizziness, lightheadedness, feeling faint, or feeling like blacking out (OHSA #1) was confirmed by the application of a tilt-table test. This tilt-table test served two purposes: (i) determination of the systolic/diastolic BP (DBP) changes, and (ii) training the subjects to recognize the sensations associated with OHSA #1.

Eligible subjects underwent training for accurate scoring of their sensation of dizziness, lightheadedness, feeling faint, or feeling like blacking out as outlined by the OHSA #1.

Following the screening period, the subject proceeded to Visit 2 to further confirm the additional eligibility criteria prior to randomization. This visit included the completion of the Orthostatic Hypotension Questionnaire (OHQ) in which a minimum score of 4 points in OHSA #1 was required. Subjects meeting all applicable inclusion criteria and none of the applicable exclusion criteria, including confirmation of relevant criteria by the independent Enrollment Steering Committee (ESC), were randomized to receive either ampreloxetine or matching placebo for the next 4 weeks.

Following randomization and completion of study assessments, the subject received a single 10-mg dose of ampreloxetine (or matching placebo) once daily (QD) for the remaining double-blind treatment period.

Weekly assessments were conducted for subjects. A description of all assessments and detailed instructions for conducting subject assessments in clinic and remotely were provided to Investigators. These instructions were provided to ensure the method and conduct of each assessment was consistent across sites and subjects for both in clinic and remote visits.

Discontinuation of subjects could occur at any time. Dose stopping criteria included meeting at least one of the following conditions.

(a) A determination from the Investigator that further administration of the study medication may pose a safety concern to the subject.

(b) Sustained (at least 4 hours) SBP ≥180 mmHg or diastolic BP (DBP) ≥110 mmHg after 3 min of standing or after 5 min in the sitting position, or a sustained (at least 4 hours) SBP ≥180 mmHg or DBP ≥110 mmHg measured in the supine state (head/torso elevated at approximately 30° from horizontal position).

(c) Intolerable AE as determined by the Investigator.

(d) Subject became pregnant.

No dose reduction was permitted at any time.

Safety assessments included a physical examination, neurological examination, vital signs (body temperature, HR and BP), body weight, ECGs, safety laboratory tests (hematology, chemistry, and urinalysis), C-SSRS, and AEs. Safety was periodically reviewed by an independent data monitoring committee.

Subjects were requested to refrain from making any significant dietary changes throughout the duration of the study. Subjects were reminded to maintain an adequate fluid intake during their scheduled visits.

Subjects completing the 4-week double-blind treatment period were eligible to enroll and continue receiving study medication in a separate, subsequent study. The final study visit for those subjects who did not complete the 4-week double-blind treatment period or who chose not to continue into the subsequent study was the follow-up visit (V7). This visit was completed two weeks from the date of the last dose.

### F. Study Objectives

The primary objective of the study was to evaluate the efficacy of ampreloxetine in subjects with multiple system atrophy (MSA), Parkinson's disease (PD), or pure autonomic failure (PAF) experiencing symptomatic neurogenic orthostatic hypotension (symptomatic nOH) compared with placebo at Week 4, as measured by the change from baseline of the Orthostatic Hypotension Symptom Assessment (OHSA) Question 1 (OHSA #1) score.

The secondary objectives of the study were as follows.

(a) To evaluate the efficacy of ampreloxetine by symptom and activity assessments using OHSA and the Orthostatic Hypotension Daily Activity Scale (OHDAS).

(b) To evaluate the efficacy of ampreloxetine using the Patient Global Impression of Change (PGI-C).

(c) To evaluate the efficacy of ampreloxetine in preventing incidence of falls.

(d) To evaluate the safety and tolerability of ampreloxetine, including adverse events (AEs) and changes in blood pressure (BP), heart rate (HR), electrocardiogram (ECG), Columbia Suicide Severity Rating Scale (C-SSRS) and laboratory tests.

The exploratory objectives of the study were the following.

(a) To evaluate the efficacy of ampreloxetine using disease-specific instruments, Unified Parkinson's Disease Rating Scale (UPDRS), Parkinson's Disease Questionnaire-8 (PDQ-8), Unified Multiple System Atrophy Rating Scale (UMSARS), and Composite Autonomic Symptoms Score-31 (COMPASS-31).

(b) To evaluate the efficacy of ampreloxetine using standing blood pressure during orthostatic standing test.

(c) To evaluate the efficacy of ampreloxetine using generic quality of life assessment EuroQol-5D-5L (EQ-5D-5L).

(d) To explore the potential benefit of ampreloxetine on nondopaminergic symptoms of subjects with primary autonomic failure.

(e) To explore pharmacodynamic markers in subjects with primary autonomic failure.

(f) To inform population PK modeling through collection of sparse PK samples in subjects with primary autonomic failure.

(g) To assess caregiver burden in caring for subjects with primary autonomic failure using Burden Scale for Family Caregivers-short version (BSFC-s).

### G. Study Evaluations

The efficacy assessments for this study included: OHQ; PGI-C; and incidence of falls. The exploratory assessments included: Orthostatic standing test; PK and pharmacodynamics; Non-Motor Symptom Scale (NMSS); EQ-5D-5L; Hospital Anxiety and Depression Scale (HADS); and Burden Scale for Family Caregivers-short version (BSFC-s). For subjects with PD, the study assessments included: UPDRS; and PDQ-8; and for subjects with MSA: COMPASS-31 and UMSARS.

The safety and tolerability assessments included: physical examination; neurological examination; vital signs, including ambulatory BP; resting ECGs; safety laboratory tests, including chemistry, hematology, and urinalysis; concomitant medication; AEs; subject compliance to study treatment; and C-SSRS.

For pharmacokinetic assessments, a sparse PK sampling strategy was employed in this study where samples were taken at select study visits. Time of study medication ingestion was accurately documented the day before and the day of each study visit where PK samples were collected.

For pharmacodynamic assessments, blood samples for pharmacodynamic markers (including [dihydroxyphenylglycol] DHPG and NE) were collected after the subject had been seated for approximately 30 minutes.

### H. Statistical Methods

1. Sample Size

This study was designed with a sample size of approximately 188 subjects total randomized in a 1:1 ratio to ampreloxetine or placebo group stratified by disease type (MSA, PD, or PAF). The study was designed to enroll a minimum of 40% subjects with MSA (i.e., at least 76 subjects overall with MSA).

The Full analysis set (FAS) is defined as all randomized subjects who have received at least 1 dose of study medication and have baseline and at least 1 post baseline measurement of OHSA #1. The primary analysis occurred when all subjects in the FAS have completed the primary endpoint assessment (OHSA #1 at Week 4) and the database has been cleaned and locked. A total sample size of 170 subjects would have an overall power of 90% to detect a treatment difference of 1.5 in the primary endpoint of change from baseline in OHSA #1, assuming a standard deviation of 3.0 for both treatment groups at a 2-sided alpha level of 0.05.

Assuming a 10% dropout rate by Week 4, it was anticipated that the study would randomize approximately 188 subjects in order to achieve 170 subjects in the FAS.

2. Study Endpoints

The primary study endpoint was the change from baseline in OHSA #1 (dizziness, lightheadedness, feeling faint, or feeling like blacking out) at Week 4.

The secondary endpoints were:
Change from baseline in OHSA composite score in Weeks 1 to 4;
Change from baseline in OHDAS composite score in Weeks 1 to 4;
PGI-C at Week 4; and
Incidence of falls.

Exploratory endpoints for all subjects were:
Standing systolic blood pressure during orthostatic standing test;
Change from baseline in OHSA #1 in Weeks 1 to 3;
Change from baseline in OHSA Questions 2 through 6 in Weeks 1 to 4;
Change from baseline in OHDAS Questions 1 through 4 in Weeks 1 to 4;
Change from baseline in OHQ overall composite score in Weeks 1 to 4;
Change from baseline in OHDAS Questions 1 and 2 in Weeks 1 to 4;
Change from baseline in EQ-5D-5L at Week 4;
PK and pharmacodynamics;
Non-Motor Symptom Scale (NMSS);
Hospital Anxiety and Depression Scale (HADS); and
Burden Scale for Family Caregivers-short version (BSFC-s).

Exploratory endpoints for subjects with PD were:
Change from baseline in UPDRS at Week 4; and
Change from baseline in PDQ-8 at Week 4.

Exploratory endpoints for subjects with MSA were:
Change from baseline in COMPASS-31 at Week 4; and
Change from baseline in UMSARS at Week 4.

Safety and tolerability endpoints included:
Physical examination;
Neurological examination;
Vital signs including ambulatory BP;
Resting ECGs;
Clinical laboratory tests, including biochemistry, hematology, urinalysis;
Concomitant medication;
AEs;
Subject compliance to study treatment; and
C-SSRS.

3. Endpoint Analysis

Analyses conducted based on the FAS were based on the assigned randomized treatments. The safety analysis set was defined as all randomized subjects who have received at least one dose of study medication and subjects were analyzed according to the actual study treatments they received. Unless specified otherwise, all data were summarized by treatment group. Continuous variables were presented using descriptive statistics. Categorical variables were summarized using subject counts and percentage of subjects in corresponding categories.

The primary efficacy evaluation was the change from baseline of OHSA #1 at Week 4. Baseline was defined as Day 1 pre-dose measurement. A mixed model for repeated measures (MMRM) was fitted to compare treatments. The model included fixed effect class terms of treatment, baseline disease type (MSA, PD, PAF), week, and continuous covariate of baseline OHSA #1 score, a random subject effect, with an unstructured covariance structure using the FAS.

Least squares means and 95% confidence intervals for the differences between ampreloxetine and placebo were calculated. Missing data in the MMRM analysis was assumed as missing at random (MAR) and was not imputed for the analysis of the primary endpoint. Sensitivity analyses on the primary endpoint were conducted using multiple imputation. The primary analysis was repeated on a set of pre-specified subgroups and presented in graphical format.

The following secondary efficacy endpoints were tested via a statistical testing procedure that protected the family-wise Type I error rate at 2-sided significance level of 5% until a failure to reject the null hypothesis occurred:
Change from baseline in OHSA composite score at Week 4;
Change from baseline in OHDAS composite score at Week 4;
PGI-C at Week 4; and
Incidence of falls at Week 4.

No statistical significance was claimed after a failure to reject the null hypothesis had occurred.

Secondary efficacy endpoints involving assessment of change from baseline, such as OHSA composite score and OHDAS composite score, were analyzed in a similar fashion as the primary efficacy endpoint of change from baseline in OHSA #1.

The PGI-C was summarized as number and percentage for subjects with “better” and “no change or worse” at Week 4. Incidence of falls was summarized as number and percentage of subjects with at least one fall at Week 4. These endpoints were tested using Cochran-Mantel Haenszel chi-square tests, stratified by disease type at baseline.

For all supportive analyses including sensitivity analyses of the primary efficacy endpoint and exploratory endpoints, nominal p-values and 95% confidence intervals with no adjustment for multiplicity are provided.

Safety data were listed by subject and summarized using the frequency of event or descriptive statistical summaries, as appropriate. Summary tables were prepared for physical examination, neurological examination, vital signs (body temperature, HR and BP), body weight, ECGs, safety laboratory tests (hematology, chemistry, and urinalysis), C-SSRS, AEs and concomitant medications.

### I. Study Results

The study did not meet its primary endpoint. Subjects receiving ampreloxetine did not have a statistically significant change from baseline of the Orthostatic Hypotension Symptom Assessment (OHSA) Question 1 (OHSA #1) score at Week 4 compared with subjects receiving placebo. The majority of treatment-related adverse events were mild or moderate in severity. Serious adverse events occurred in two subjects on placebo and four on ampreloxetine and none were considered related to the study drug; no deaths were reported. There was no signal for supine hypertension.

However, it was also shown that at Week 4, subjects with MSA who were administered ampreloxetine showed decrease in Anxiety Total Score from baseline, indicating effectiveness of ampreloxetine against anxiety due to nOH. Table 5 shows changes of Anxiety Total Score for MSA subjects at Week 4 from baseline. Anxiety Total Score was determined using standard procedures (see, e.g., Rishi et al. Hospital anxiety and depression scale assessment of 100 patients before and after using low vision care: A prospective study in a tertiary eye-care setting. Indian J Ophthalmol. 2017 November; 65(11):1203-1208).

TABLE 5

| | Change from Baseline | | | |
|---|---|---|---|---|
| | Placebo | | Ampreloxetine 10 mg | |
| | (N = 90) | | (N = 90) | |
| N | 31 | | 33 | |
| Mean (SD) | 0.26 | (2.543) | −0.67 | (3.533) |
| Median | 0.00 | | −1.00 | |
| Q1, Q3 | −1.00, 2.00 | | −2.00, 1.00 | |
| Min, Max | −6.0, 4.0 | | −8.0, 10.0 | |
| LS Mean (SE) | 0.34 | (0.518) | −0.75 | (0.502) |
| LS Mean Difference (SE) | | | −1.09 | (0.722) |
| 95% CI for LS Mean | | | (−2.54, 0.35) | |
| Difference | | | | |

| | |
|---|---|
| P-value vs. Placebo | 0.136 |

### Example 7

A Phase 3 22-Week Clinical Study in Subjects with Symptomatic nOH

This was a Phase 3, multi-center, randomized withdrawal study to evaluate the sustained benefit in efficacy and safety of ampreloxetine (administered as ampreloxetine hydrochloride) in subjects with primary autonomic failures (MSA, PD, or PAF) and symptomatic nOH after 22 weeks of treatment. Eligible subjects were either (i) completers of the study of Example 6 (“Completers Group”), or (ii) symptomatic nOH subjects meeting all applicable study inclusion criteria and none of the applicable exclusion criteria (“De Novo Group”).

The study consisted of 3 periods: (i) 16-week open-label (OL) treatment with ampreloxetine, (ii) 6-week randomized placebo-controlled treatment, and (iii) 2-week follow-up (only for subjects who do not enroll in continuing Study 0171). A 4-week screening period applied to the De Novo Group only and the Screening visit was conducted in clinic for all De Novo Group subjects.

A total of 203 subjects entered this study (170 subjects from Example 6 (85 from the ampreloxetine group and 85 from the placebo group) and 33 de novo subjects). During the OL period, 55 subjects discontinued treatment. At 16 weeks, 64 subjects were randomized to the ampreloxetine group (6 subjects discontinued the study) and 64 subjects were randomized to the placebo group (3 subjects discontinued the study). A total of 119 subjects completed the study. Of the 128 subjects entering the randomized period, 40 subjects had MSA (31%); 68 subjects had PD (53%); and 20 subjects had PAF (16%) (in each category, the subjects were split equally between ampreloxetine and placebo).

### A. Study Population

This study enrolled adult subjects with confirmed symptomatic nOH due to MSA, PD, or PAF, who met all of the inclusion criteria and none of the exclusion criteria defined in the study protocol.

1. Subjects Entering from Completers Group

Following signing of the informed consent, subjects entered Visit 1 (V1), which was conducted on the same day as Visit 6 (V6/D29) of Example 6. The visit modality for this example, either in-clinic or remote for each subject, for a given subject was consistent with the modality selected by the subject for Example 6. Example 6 procedures conducted at V6 served as the baseline assessments for V1 of this example.

Beginning on Day 2, subjects received a single dose of 10 mg ampreloxetine once daily (QD) and continued thereafter for the 16-week duration of the OL treatment period. Following this 16-week OL treatment period, subjects were randomized to either continue on the active treatment or placebo (“PBO”) for a period of 6 weeks.

2. Subjects in De Novo Group

Eligible subjects underwent training of accurate scoring of their sensation of dizziness, lightheadedness, feeling faint, or feeling like blacking out, as outlined by Orthostatic Hypotension Symptom Assessment Question 1 (OHSA #1).

Following the screening period, subjects proceeded to V1 to further confirm the additional eligibility criteria prior to proceeding. This included the completion of the Orthostatic Hypotension Questionnaire (OHQ) in which a minimum score of 4 points in OHSA #1 was required.

Subjects meeting all inclusion criteria, none of the exclusion criteria, and whose disease characterization was confirmed by the independent Enrollment Steering Committee (ESC) received ampreloxetine in the OL period.

Beginning on Day 2, subjects received a single dose of 10 mg ampreloxetine once daily (QD) and continued thereafter for the 16-week duration of the OL treatment period. Following this 16-week OL treatment period, subjects were randomized to either continue on the active treatment or PBO for a period of 6 weeks.

3. Inclusion Criteria

Subjects from the Completers Group who met the following criteria were eligible for study enrollment.

(a) Completed 4 weeks of double-blind treatment in Example 6 (V6) and, in the opinion of the Investigator, could benefit from continued treatment with ampreloxetine. No minimum score of OHSA #1 was required to enter V1.

(b) The subject had a minimum of 80% study medication compliance in Example 6.

(c) The subject was able to understand the nature of the study and provide written informed consent prior to the conduct of any study procedures (including an understanding that entry to this study may result in changes occurring in the subject's current therapeutic regimen).

(d) The subject was willing to continue on treatment regardless of the possibility of randomization to either ampreloxetine or PBO during the randomized withdrawal phase and continued to meet the inclusion criteria for the preceding study (Example 6) with the exception that tilt-table test, ESC review and approval of eligibility were required for entry.

Subjects from the De Novo Group who met the inclusion criteria for Example 6 were eligible for study enrollment in this study.

4. Exclusion Criteria

Subjects from the Completers Group who met any of the following criteria were not eligible for study enrollment.

(a) Subject may not be enrolled in another clinical trial (other than Example 6).

(b) Subject has psychiatric, neurological, or behavioral disorders that may interfere with the ability of subjects to give informed consent or interfere with the conduct of the study.

(c) Medical, laboratory, or surgical issues deemed by the Investigator to be clinically significant.

(d) Uncooperative attitude or reasonable likelihood of non-compliance with the protocol.

(e) Subject has a concurrent disease or condition that, in the opinion of the Investigator, would confound or interfere with study participation or evaluation of safety, tolerability, or pharmacokinetics of the study drug.

Subjects from the De Novo Group who met the exclusion criteria for Example 6 were not eligible for study enrollment in this study.

5. Prohibitions and Restrictions

The same prohibitions and restrictions used for study participation for Study Example 6 were used.

### B. Test Product, Dose, and Route of Administration: Regimen: Duration of Treatment

1. For OL Treatment Period

All subjects received ampreloxetine and continued to take the study medication once daily starting on Day 2 through the end of the treatment period. The test product ampreloxetine supplied as a 10 mg tablet in 35-count high-density polyethylene bottles.

2. For Randomized Treatment Period

Subjects randomized to ampreloxetine received ampreloxetine tablets and continued to take the study medication once daily starting in the morning post randomization (Day 2) through the end of the treatment period. The test product ampreloxetine supplied as a 10-mg tablet in 35-count or 5-count high-density polyethylene bottles labeled in a blinded fashion (i.e., bottle label for test product was indistinguishable from reference product except for a coded, unique bottle number).

3. For Both Treatment Periods

Ampreloxetine was administered orally without regard to food at approximately the same time each morning and was taken with approximately 8 ounces of water.

### C. Reference Product, Dose, and Route of Administration: Regimen: Duration of Treatment

1. For OL Treatment Period

No subjects received the reference product during the OL treatment period.

2. For Randomized Treatment Period

Subjects randomized to PBO received PBO tablets that match ampreloxetine tablets in excipient content (except for ampreloxetine), appearance, tablet count, and in packaging (e.g., bottle label for reference product was indistinguishable from test product except for a coded, unique bottle number).

The PBO tablet was administered orally without regard to food at approximately the same time each morning and was taken with approximately 8 ounces of water.

### D. Study Execution

1. Open Label Period (Weeks 1 to 16)

Subjects enrolled into the OL period of the study had visits scheduled for Day 15, Day 29, and every 4 weeks thereafter for assessments as outlined in the Schedule of Study Procedures. At V3 (Week 4), following the initial 4-week OL treatment, subjects had to demonstrate a reduction in OHSA #1 of at least 2 points compared to the baseline value, as determined in Example 6 for subjects entering from Example 6 and from V1 for de novo subjects, in order to continue in this study. Those subjects not meeting this continuation criterion were discontinued and underwent an end of study visit. The end of study visit was completed within 2 weeks from the date of the last dose.

All subjects completing the initial 4-week OL treatment period and meeting the continuation criterion continued receiving open label ampreloxetine for 12 additional weeks (16 weeks total).

2. Double-Blind Period (Weeks 17 to 22)

Following the completion of a total of 16 weeks OL treatment with ampreloxetine (V6), subjects were assessed for randomization in a 1:1 manner. Eligible subjects received 6 weeks of double-blind treatment of ampreloxetine or PBO once daily. Only subjects with OHSA #1 score of ≤7 were eligible for randomization for the double-blind treatment period.

3. Treatment Period (Weeks 1 to 22)

No dose reduction was permitted during this treatment period. Subjects unable to tolerate 10 mg ampreloxetine were discontinued from the study. At any time during the study, if a subject met at least one of the following stopping rules, they were discontinued and underwent an end of study visit:
(a) A determination from the PI that further administration of the investigational product may pose a safety concern to the subject;
(b) Sustained (at least 4 hours) SBP ≥180 mmHg or diastolic BP (DBP) ≥110 mmHg after 3 min of standing or after 5 mins in the sitting position, or a sustained (at least 4 hours) SBP ≥180 mmHg or DBP ≥110 mmHg measured in the supine state (head/torso elevated at approximately 300 from horizontal position);
(c) Intolerable AE, as determined by the PI; or
(d) Subject becomes pregnant.

Safety assessments included a physical examination, neurological examination, vital signs (body temperature, HR, respiratory rate, and blood pressure), body weight, 12-lead electrocardiograms, laboratory tests (hematology, chemistry, and urinalysis), Columbia Suicide Severity Rating Scale (C-SSRS) and monitoring of AEs. Safety was periodically reviewed by an Independent Data Monitoring Committee (IDMC).

Subjects were requested to refrain from making any significant dietary changes throughout the duration of the study. During their scheduled visits, subjects were reminded to maintain an adequate fluid intake.

Subjects completing the 6-week double-blind treatment period were eligible to continue into the OL, long term safety study (Study 0171). Those subjects who did not complete the 6-week double-blind treatment period or who chose not to continue into Study 0171 completed the Early Termination Visit (V9) or Follow-up visit (V10), respectively. The Follow-Up Visit was completed 2 weeks from the date of the last dose.

4. Continuation Criteria

At V3 (Week 4), following the initial 4-week OL treatment, subjects must demonstrate a reduction in OHSA #1 of at least 2 points compared to the baseline value, as determined in Example 6 for subjects entering from Example 6 and from V1 for de novo subjects, in order to continue in this study.

5. Randomization Criteria for Double-Blind Period

The following randomization criteria were used for the double-blind period.

(a) Subject had OHSA #1 score of <7.

(b) Subject's unused OL study medications (10 mg ampreloxetine tablets) were returned to site.

(c) The subject had a minimum of 80% study medication compliance in OL treatment period.

(d) Subjects with excessive deterioration of disease or symptoms during the OL phase and who in the opinion of the Investigator, would not benefit from continued participation in the study were excluded.

### E. Study Evaluations

The efficacy assessments for this study included: OHQ and PGI-S; and subject's symptomatic improvement as measured by a wearable device. Exploratory assessments included: orthostatic standing test; NMSS; EQ-5D-5L; HADS; and BSFC-s. For subjects with PD, exploratory assessments included: Unified Parkinson's Disease Rating Scale (UPDRS); and Parkinson's Disease Questionaire-8 (PDQ-8). For subjects with MSA, exploratory assessments included: Unified Multiple System Atrophy Rating Scale (UMSARS); and Composite Autonomic Symptom Score-31 (COMPASS-31).

Safety and tolerability assessments for this study included: physical examination including weight; neurological examination; vital signs, including ambulatory BP; resting ECGs; clinical laboratory assessments, including serum chemistry, hematology, and urinalysis; concomitant medications; AEs; and C-SSRS.

A sparse PK sampling strategy was employed in this study where samples were taken at select study visits as defined in the Schedule of Study Procedures. Time of ingestion of study medication was accurately documented the day before and the day of each study visit when PK samples were collected.

Blood samples for pharmacodynamic markers ([dihydroxyphenylglycol] DHPG and NE) were collected at select study visits as defined in the Schedule of Study Procedures after the subject had been seated for 30 minutes. This was applicable to all consenting subjects.

### F. Statistical Methods

1. Sample Size

A total planned sample size of 154 at randomization was designed to provide 90% power to detect a difference of 0.25 (0.25 ampreloxetine vs. 0.50 placebo) in the proportion of subjects that meet the criteria of treatment failure at Week 6 (V9, D155) during the double-blind randomized withdrawal phase at the 0.05 significance level using a 2-sided test. The actual total sample size at randomization was 128.

2. Study Endpoints

The primary study endpoint was the proportion of treatment failure at Week 6 during the double-blind randomized withdrawal phase. Treatment failure is defined as subjects who meet the following criteria at Week 6 following randomization (V9, D155): change (worsening) from baseline in OHSA #1 score of 1.0 point and worsening of disease severity as assessed by a 1-point change in PGI-S.

The assessments done at the Week 16 (V6, D113) visit in the OL phase prior to randomization were considered baseline for the double-blind randomized withdrawal phase of the study. Subjects who withdrew for any reason prior to V9 (D155) or subjects who failed to provide assessment at V9 (D155) were considered as treatment failures.

The secondary endpoints included:
(a) Change from baseline in OHSA #1 at Week 6 post randomization (V9, D155);
(b) Change from baseline in OHSA composite score at Week 6 post randomization (V9, D155);
(c) Change from baseline in OHDAS composite score at Week 6 post randomization (V9, D155);
(d) Change from baseline in PGI-S at Week 6 post randomization (V9, D155);
(e) Change from baseline in percentage of time spent in standing position as measured by a wearable device at Week 6 post randomization (V9, D155); and
(f) Change from baseline in average number of steps taken as measured by a wearable device at Week 6 post randomization (V9, D155).

Exploratory endpoints included:
(a) Standing SBP during orthostatic standing test at Week 6 post randomization (V9, D155);
(b) Change from baseline in OHQ overall composite score at Week 6 post randomization (V9, D155);
(c) Change from baseline in EQ-5D-5L at Week 6 post randomization (V9, D155);
(d) NMSS at Week 6 post randomization (V9, D155);
(e) HADS at Week 6 post randomization (V9, D155); and
(f) BSFC-s at Week 6 post randomization (V9, D155).

For subjects with PD, the exploratory endpoints included:
(a) Change from baseline in UPDRS at Week 6 post randomization (V9, D155); and
(b) Change from baseline in PDQ-8 at Week 6 post randomization (V9, D155).

For subjects with MSA, the exploratory endpoints included:
(a) Change from baseline in UMSARS at Week 6 post randomization (V9, D155); and
(b) Change from baseline in COMPASS-31 at Week 6 post randomization (V9, D155).

Safety and tolerability endpoints included:
(a) Physical examination;
(b) Neurological examination;
(c) Vital signs including ambulatory BP;
(d) Resting ECGs;
(e) Clinical laboratory assessments including biochemistry, hematology, urinalysis;
(f) Concomitant medication;
(g) Adverse events (AEs); and
(h) Columbia Suicide Severity Rating Scale (C-SSRS).

Exploratory safety and tolerability endpoints included:
(a) PK and pharmacodynamic parameters.

3. Endpoint Analysis

All efficacy analyses in the double-blind randomized withdrawal phase were performed based on the Full Analysis Set (FAS) using the assigned randomized treatments. The FAS of the double-blind randomized withdrawal phase was defined as all randomized subjects who have received at least 1 dose of study medication following randomization.

The safety analysis set of the double-blind randomized withdrawal phase was identical.

The FAS and safety analysis set for the OL phase were identical and were defined as all enrolled subjects who received at least 1 dose of ampreloxetine.

In the double-blind randomized withdrawal phase, all data were summarized by treatment group. In the OL phase, all data were summarized overall and by enrollment group (Placebo Example 6 Completers, Ampreloxetine Example 6 Completers, and De Novo Group). Continuous variables were summarized using descriptive statistics. Categorical variables were summarized using subject counts and percentage of subjects in corresponding categories.

The primary study endpoint was the proportion of treatment failure at Week 6 during the double-blind randomized withdrawal phase. Treatment failure was defined as subjects who met the following criteria at Week 6 following randomization (V9, D155): change (worsening) from baseline in OHSA #1 score of 1.0 point and worsening of disease severity as assessed by a 1-point change in PGI-S.

The assessments done at the Week 16 (V6, D113) visit in the OL phase prior to randomization were considered baseline for the double-blind randomized withdrawal phase of the study. Subjects who withdrew for any reason prior to V9 (D155) or subjects who fail to provide assessment at V9 (D155) were counted as treatment failures.

A logistic regression model was fitted to estimate the effect of treatment on the primary endpoint. The model included terms for treatment and baseline disease type (MSA, PAF, PD) and also included terms for baseline OHSA #1 score and baseline PGI-S as continuous covariates. Point estimates and 95% CIs for odds ratios were presented. Also presented were “least squares” treatment failure proportions (point estimates and standard errors) for each treatment and disease type. In addition, the proportions of subjects with at least a 1-point worsening in OHSA #1 and the proportions of subjects with at least a 1-point worsening in PGI-S were summarized.

The primary analysis was repeated for the per-protocol analysis set as defined in the Statistical Analysis Plan (SAP).

To assess consistency of treatment failure ampreloxetine:placebo odds ratios across subgroups, the analysis of the primary endpoint was repeated for each of the subgroups specified in the SAP: MSA, PD, and PAF subjects; men and women; and subjects with baseline NE <200 pg/mL and ≥200 pg/mL. A forest plot was provided showing the ampreloxetine:placebo odds ratio point estimates and 95% CIs for each subgroup.

The secondary endpoints were analyzed by fitting a mixed model for repeated measures with terms for treatment, disease type (MSA, PD, PAF), visit, and treatment by visit interactions, and the baseline value of the endpoint as a continuous covariate. Within-subject correlation was modelled using an unstructured variance-covariance matrix. The Kenward and Roger method for approximating the denominator degrees of freedom was used. Missing endpoint values were assumed to be missing at random (MAR). Least squares means and 95% confidence intervals for the differences between ampreloxetine and placebo were calculated and presented.

Exploratory endpoints involving assessment of change from baseline at multiple time points such as standing SBP during orthostatic standing test were analyzed similarly.

Exploratory endpoints involving assessment of change from baseline but only at Week 6 post randomization (V9, D155) such as PDQ-8, UPDRS, UMSARS, and COMPASS-31 were analyzed by fitting an analysis of covariance model with terms for treatment, baseline disease type (MSA, PD, PAF), and the baseline score for the endpoint as a continuous covariate.

A multiple testing plan was used to control the family-wise error type 1 error rate for the primary and secondary endpoints at 0.05. If the primary endpoint was met, i.e., the ampreloxetine vs. placebo odds of treatment failure at Week 6 post randomization (V9, D155) point estimate was <1 and the p-value was <0.05, secondary efficacy endpoints were tested in the sequence shown below until the first failure to reject a null hypothesis occurred:
1. Change from baseline in OHSA #1 at Week 6 post randomization (V9, D155);
2. Change from baseline in OHSA composite score at Week 6 post randomization (V9, D155);
3. Change from baseline in OHDAS composite score at Week 6 post randomization (V9, D155);
4. Change from baseline in PGI-S at Week 6 post randomization (V9, D155)
5. Change from baseline in percentage of time spent in standing position (including walking) as measured by a wearable device at Week 6 post randomization (V9, D155);
6. Change from baseline in average number of steps taken as measured by a wearable device at Week 6 post randomization (V9, D155).

For all other analyses, there were no adjustments for multiplicity.

Safety data were listed by subject and summarized descriptively. Summaries and listings were presented separately for the OL phase and the double-blind randomized withdrawal phase. Summary tables were provided for vital signs, body weight, ECGs, safety laboratory tests, C-SSRS, AEs, and concomitant medications.

### G. Study Results

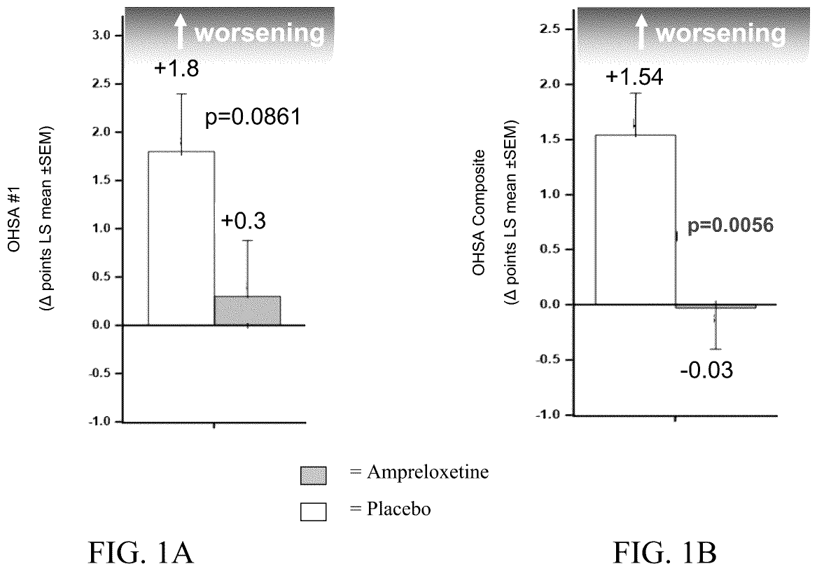

In subjects having multiple system atrophy and symptomatic neurogenic orthostatic hypotension, it has now been discovered that treatment with ampreloxetine for 22 weeks surprisingly results in a measurable reduction in both (i) the subject's symptoms of dizziness, lightheadedness, feeling faint, or feeling like he or she might black out as determined using the subject's OHSA item 1 score and (ii) the subject's overall impression of symptom severity as determined using the subject's OHSA composite score. For example, FIG. 1A illustrates that the OHSA item 1 score for MSA subjects administered a placebo worsens (+1.8) compared to subjects administered ampreloxetine (+0.3; p=0.0861) at the end of the 6-week double-blind randomized withdrawal period; and FIG. 1B illustrates that the OHSA composite score for MSA subjects administered a placebo worsens significantly (+1.54) compared to subjects administered ampreloxetine (−0.03; p=0.0056) during the 6-week double-blind randomized withdrawal period.

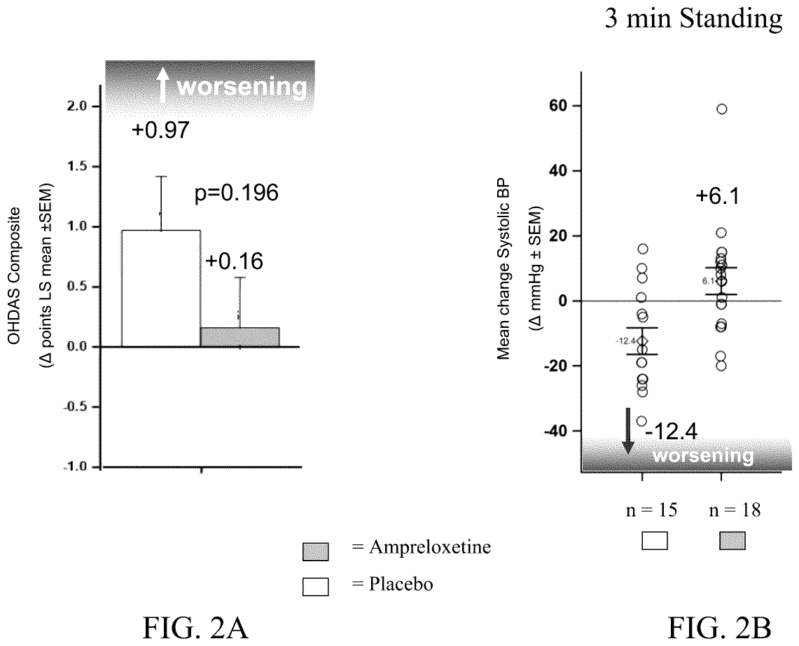

In subjects having multiple system atrophy and symptomatic neurogenic orthostatic hypotension, it has also now been discovered that treatment with ampreloxetine for 22 weeks surprisingly results in a measurable reduction in the subject's Orthostatic Hypotension Daily Activity Scale (OHDAS) composite score and a measurable increase in the subject's systolic blood pressure upon standing for 3 minutes. For example, FIG. 2A illustrates that the OHDAS composite score for MSA subjects administered a placebo worsens significantly (+0.97) compared to subjects administered ampreloxetine (+0.16; p=0.196) during the 6-week double-blind randomized period for Example 7. FIG. 2B illustrates that the systolic blood pressure upon standing for 3 minutes for MSA subjects administered a placebo worsens significantly (−12.4 mmHg) compared to subjects administered ampreloxetine (+6.1) during the 6-week double-blind randomized period.

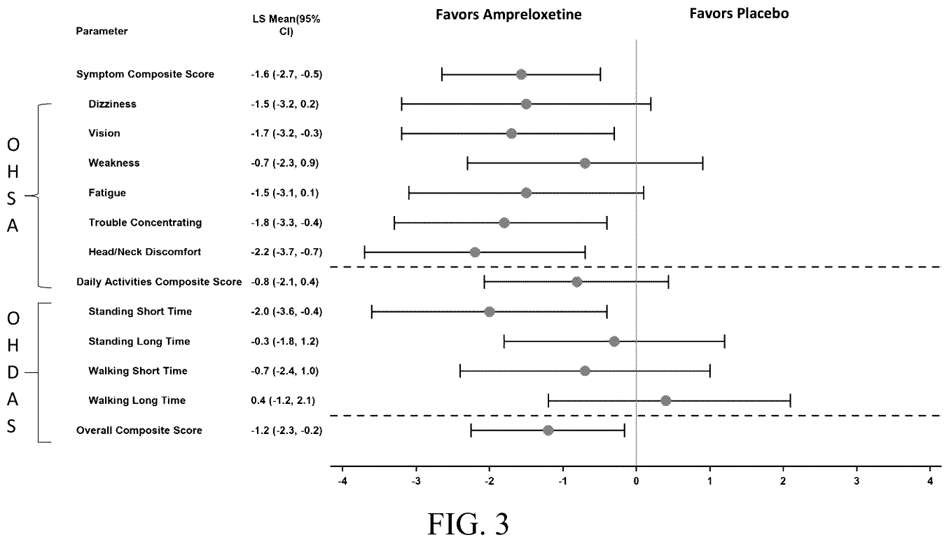

FIG. 3 illustrates that the OHSA and OHDAS composite scores and individual subscores for symptoms and daily activities for MSA subjects all favor ampreloxetine at the end of the 6-week double-blind randomized period, except for OHDAS walking long time which favor placebo.

The OHSA item 1 score is an OHQ item score and the OHSA and OHDAS composite scores are OHQ subscale scores (see Appendix 1 and, e.g., Kaufmann et al., Clin. Auton. Res. (2012), 22:79-90).

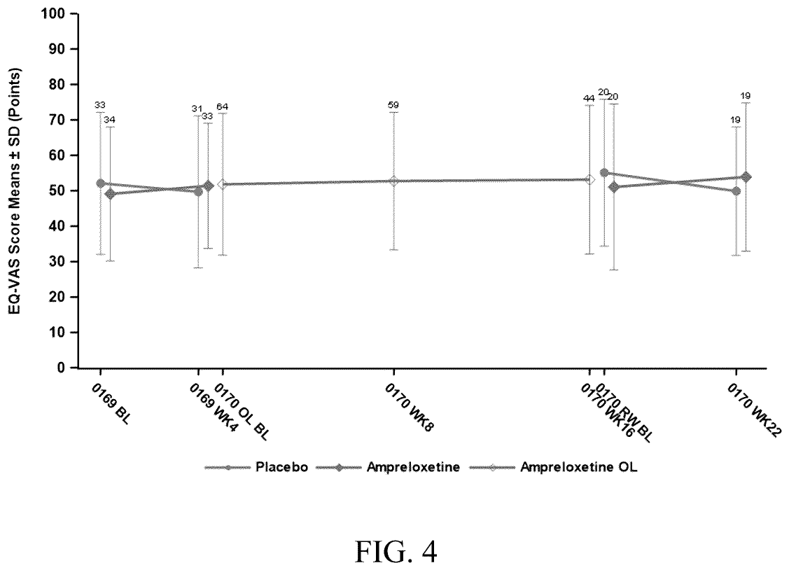

Additionally, in subjects with multiple system atrophy, it has now been discovered that the magnitude of decline in the quality of life may be reduced when the subject is treated with ampreloxetine. For example, FIG. 4 indicates that quality of life declines in MSA subjects administered a placebo whereas quality of life declines less or improves in subjects administered ampreloxetine. Quality of life was measured using the EQ-5D-5L visual analog scale (see Appendix 2; and e.g., McCaffrey et al. Health and Quality of Life Outcomes (2016) 14:133).

Moreover, it has also been discovered that the supine norepinephrine level in a subject having multiple system atrophy continues to increase for at least about 8 weeks when the subject is treated daily with ampreloxetine as shown in Table 8:

TABLE 8

| | | | | NE at End of Ex. | | NE on Day 29 | |
|---|---|---|---|---|---|---|---|
| Example 6 | Underlying | NE at Start of | | 6/Start of Ex. 7 | | of Ex. 7 Open | |
| Group | Disease | Ex. 6 (pg/ml) | | (pg/mL) | | Label (pg/mL)* | |
| Placebo | MSA | 331 ± 213 | (27) | 291 ± 173 | (22) | 557 ± 298 | (13) |
| | PAF | 186 ± 83 | (13) | 156 ± 82 | (13) | 267 ± 87 | (7) |
| | PD | 353 ± 229 | (34) | 340 ± 266 | (29) | 443 ± 157 | (21) |
| Ampreloxetine | MSA | 280 ± 138 | (25) | 439 ± 250 | (20) | 539 ± 361 | (10) |
| 10 mg QD | PAF | 232 ± 110 | (11) | 357 ± 204 | (11) | 455 ± 238 | (3) |
| | PD | 340 ± 237 | (32) | 407 ± 154 | (25) | 552 ± 270 | (25) |

*All subjects received ampreloxetine in the open-label part. NE (pg/ml) presented as mean ± SD (n).

Table 8 shows that supine norepinephrine (NE) levels in subjects with MSA continue increasing beyond 4 weeks of treatment with ampreloxetine. Before treatment with ampreloxetine, supine norepinephrine (NE) levels in subjects with MSA are less than about 350 pg/mL. After about 8 weeks of treatment, supine norepinephrine levels are greater than about 500 pg/mL. Supine norepinephrine levels were determined using standard procedures (see, e.g., Goldstein et al., Annals of Neurology, 1989:26(4) 558-563; and Palma et al., Neurology, 2018:91(16) e1539-e1544).

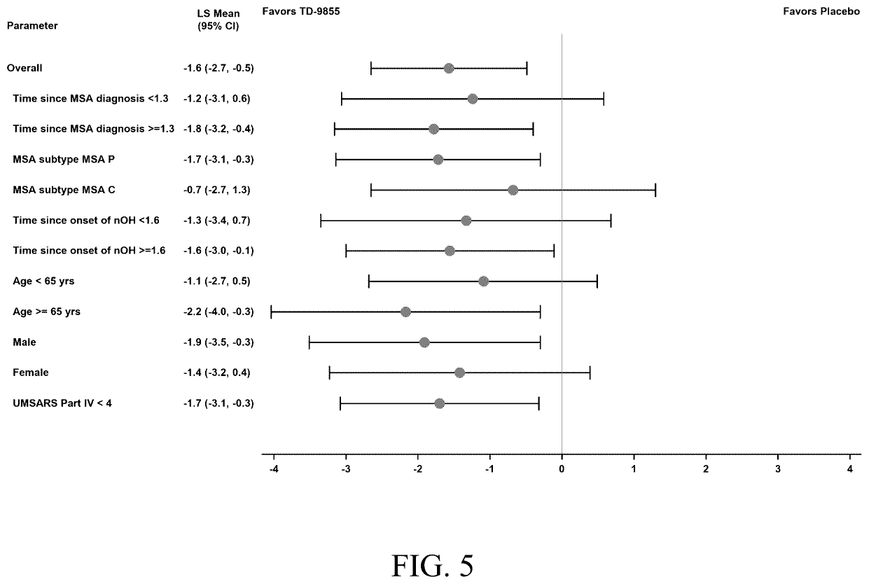

OHSA and OHDAS scores at week 22 (the end of the 6-week double-blind randomized period) were analyzed for subgroups of MSA subjects, and it was shown that ampreloxetine was favored across substantially all subgroups of MSA subjects. For example, FIG. 5 and Table 9 illustrates that the OHSA composite scores for subgroups of MSA subjects all favored ampreloxetine at week 22. The subgroups include MSA-parkinsonian type (MSA-P), MSA-Cerebellar Ataxia (MSA-C), nOH onset less than 1.6 years before the start of the study (nOH onset <1.6 years), nOH onset 1.6 years or more before the start of the study (nOH onset >=1.6 years), MSA diagnosis within less than 1.3 years before the start of the study (MSA diagnosis <1.3 years), MSA diagnosis within 1.3 years or more before the start of the study (MSA diagnosis >=1.3 years), Unified Multiple System Atrophy Rating Scale (UMSARS) part 4 less than 4 (UMSARS Part IV <4), UMSARS Part IV 4 or more (UMSARS Part IV >=4), male, female, less than 65 years old (<65 years), and 65 years old or older (>=65 years).

TABLE 9

| | | | | | | LS Mean |
|---|---|---|---|---|---|---|
| | | | | | LS | Differ- |
| Subgroup | | N | Mean | Median | Mean | ence |
| Overall | Placebo | 20 | 1.48 | 1.45 | 1.54 | |
| | Ampreloxetine | 20 | −0.05 | 0.05 | −0.03 | −1.57 |
| MSA-P | Placebo | 11 | 1.06 | 1.35 | 1.22 | |
| | Ampreloxetine | 12 | −0.39 | 0.05 | −0.50 | −1.72 |
| MSA-C | Placebo | 9 | 2.01 | 1.95 | 1.78 | |
| | Ampreloxetine | 8 | 0.76 | 0.50 | 1.10 | −0.68 |
| nOH onset <1.6 | Placebo | 11 | 1.80 | 1.70 | 1.82 | |
| years | Ampreloxetine | 8 | 0.43 | 0.50 | 0.48 | −1.33 |
| nOH onset | Placebo | 9 | 0.96 | 0.75 | 1.11 | |
| >=1.6 years | Ampreloxetine | 12 | −0.41 | −0.20 | −0.45 | −1.56 |
| MSA diagnosis | Placebo | 10 | 2.08 | 2.35 | 1.97 | |
| <1.3 years | Ampreloxetine | 9 | 0.67 | 0.70 | 0.73 | −1.24 |
| MSA diagnosis | Placebo | 10 | 1.01 | 0.95 | 1.11 | |
| >=1.3 years | Ampreloxetine | 11 | −0.64 | −0.10 | −0.67 | −1.78 |
| UMSARS Part | Placebo | 12 | 1.78 | 1.70 | 1.71 | |
| IV <4 | Ampreloxetine | 18 | −0.04 | 0.25 | 0.01 | −1.70 |
| UMSARS Part | Placebo | 8 | 1.01 | 0.7 | 1.27 | |
| IV >=4 | Ampreloxetine | 2 | −0.15 | −0.15 | −0.17 | −1.44 |
| Male | Placebo | 13 | 1.53 | 1.6 | 1.4874 | |
| | Ampreloxetine | 8 | −0.46 | 0.20 | −0.3923 | −1.8762 |
| Female | Placebo | 7 | 1.40 | 1.3 | 1.4839 | |
| | Ampreloxetine | 12 | 0.23 | 0.00 | 0.2078 | −1.3996 |
| <65 Years | Placebo | 12 | 1.31 | 1.40 | 1.65 | |
| | Ampreloxetine | 9 | 0.8 | 0.4 | 0.55 | −1.09 |
| >=65 Years | Placebo | 8 | 1.76 | 1.50 | 1.57 | |
| | Ampreloxetine | 11 | −0.75 | −0.70 | −0.60 | −2.17 |

It was surprising that ampreloxetine was able to treat or alleviate symptomatic nOH in severe, advanced, or later stage MSA patients, such as subjects who had nOH onset 1.6 years or more before the start of the study, MSA diagnosis within 1.3 years or more before the start of the study, MSA diagnosis within less than 1.3 years before the start of the study, or UMSARS Part IV score of 4 or more.

Table 10 shows that the OHDAS item 1 (activity that requires standing for a short time) scores for subgroups of MSA subjects mostly favored ampreloxetine at week 22. The subgroups include MSA-parkinsonian type (MSA-P), MSA-Cerebellar Ataxia (MSA-C), nOH onset less than 1.6 years before the start of the study (nOH onset <1.6 years), nOH onset 1.6 years or more before the start of the study (nOH onset >=1.6 years), MSA diagnosis within less than 1.3 years before the start of the study (MSA diagnosis <1.3 years), MSA diagnosis within 1.3 years or more before the start of the study (MSA diagnosis >=1.3 years), Unified Multiple System Atrophy Rating Scale (UMSARS) part 4 less than 4 (UMSARS Part IV <4), male, female, less than 65 years old (<65 years), and 65 years old or older (>=65 years).

TABLE 10

| | | | | | LS | LS Mean |
|---|---|---|---|---|---|---|
| Subgroup | | N | Mean | Median | Mean | Difference |
| Overall | Placebo | 20 | 1.2 | 1.5 | 1.6 | |
| | Ampreloxetine | 20 | −0.2 | 0.0 | −0.4 | −2.0 |
| MSA-P | Placebo | 11 | 0.6 | 1.0 | 1.5 | |
| | Ampreloxetine | 12 | −0.7 | 0.0 | −1.2 | −2.7 |
| MSA-C | Placebo | 9 | 1.8 | 1.5 | 1.9 | |
| | Ampreloxetine | 8 | 0.7 | 0.0 | 0.9 | −1.0 |
| nOH onset <1.6 | Placebo | 11 | 2.1 | 3.0 | 2.1 | |
| years | Ampreloxetine | 8 | −0.3 | 0.0 | −0.3 | −2.4 |
| nOH onset | Placebo | 9 | −0.3 | 0.0 | 0.5 | |
| >=1.6 years | Ampreloxetine | 12 | −0.1 | 0.0 | −0.3 | −0.8 |
| MSA diagnosis | Placebo | 10 | 3.0 | 3.0 | 2.8 | |
| <1.3 years | Ampreloxetine | 9 | 0.1 | 0.0 | 0.7 | −2.7 |
| MSA diagnosis | Placebo | 10 | −0.6 | 0.0 | 0.1 | |
| >=1.3 years | Ampreloxetine | 11 | −0.4 | 0.0 | −0.6 | −0.7 |
| UMSARS Part | Placebo | 12 | 2.5 | 2.0 | 2.5 | |
| IV <4 | Ampreloxetine | 18 | −0.3 | 0.0 | −0.3 | −2.8 |
| UMSARS Part | Placebo | 8 | −1.8 | −2.0 | −1.4 | |
| IV >=4 | Ampreloxetine | 2 | 1.0 | 1.0 | 0.5 | 1.9 |
| Male | Placebo | 13 | 1.6 | 2.0 | 1.7 | |
| | Ampreloxetine | 8 | −0.8 | 0.0 | −1.2 | −2.8 |
| Female | Placebo | 7 | 0.0 | 0.0 | 1.1 | |
| | Ampreloxetine | 12 | 0.3 | 0.0 | 0.2 | −1.0 |
| <65 Years | Placebo | 12 | 1.7 | 2.0 | 2.4 | |
| | Ampreloxetine | 9 | 0.0 | 0.0 | −0.5 | −2.9 |
| >=65 Years | Placebo | 8 | 0.6 | 1.0 | 0.6 | |
| | Ampreloxetine | 11 | −0.3 | 0.0 | −0.4 | −1.0 |

Again, it was surprising that ampreloxetine was able to treat or alleviate symptomatic nOH in relatively severe, advanced or later stage MSA patients, such as subjects who had nOH onset 1.6 years or more before the start of the study, MSA diagnosis within 1.3 years or more before the start of the study, MSA diagnosis within less than 1.3 years before the start of the study, or UMSARS Part IV score of 4 or more.

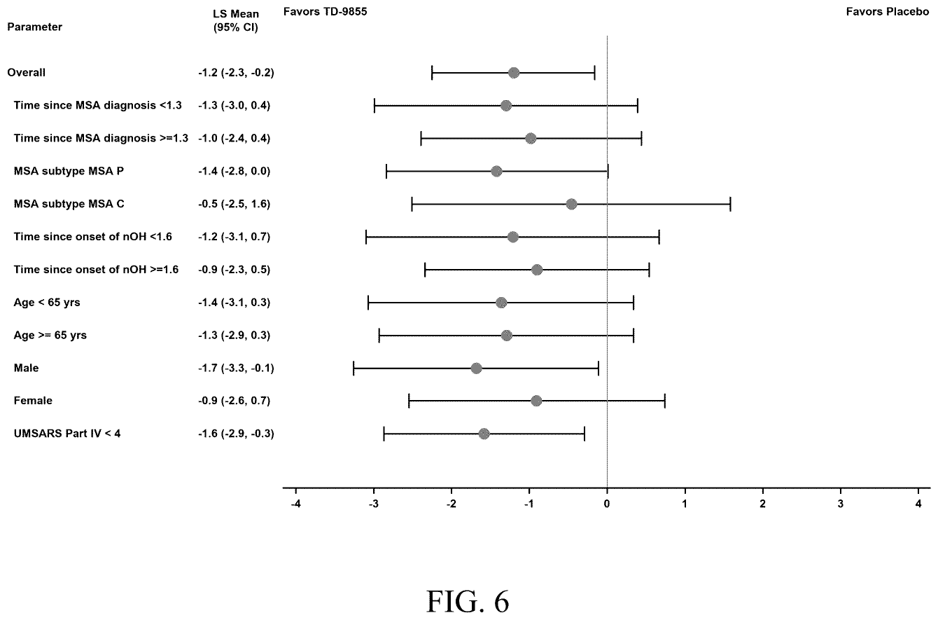

FIG. 6 illustrates that the OHQ composite scores for subgroups of MSA subjects all favored ampreloxetine at week 22 (the end of the 6-week double-blind randomized period). The subgroups include MSA-parkinsonian type (MSA-P), MSA-Cerebellar Ataxia (MSA-C), nOH onset less than 1.6 years before the start of the study (nOH onset <1.6 years), nOH onset 1.6 years or more before the start of the study (nOH onset >=1.6 years), MSA diagnosis within less than 1.3 years before the start of the study (MSA diagnosis <1.3 years), MSA diagnosis within 1.3 years or more before the start of the study (MSA diagnosis >=1.3 years), Unified Multiple System Atrophy Rating Scale (UMSARS) part 4 less than 4 (UMSARS Part IV <4), male, female, less than 65 years old (<65 years), and 65 years old or older (>=65 years).

The subgroup analysis also demonstrates that ampreloxetine was surprisingly beneficial for subjects of different baseline severity levels of nOH, despite MSA being a progressive condition. Table 11 shows changes of OHSA composite scores for subgroups of MSA subjects at week 22 (the end of the 6-week double-blind randomized period), where each subgroup had different degree of nOH, as shown by OHSA composite scores or OHSA item 1 scores, before double-blind randomized period (RW baseline), or before treatment (pre treatment baseline).

TABLE 11

| | | | | | | LS |
|---|---|---|---|---|---|---|
| | | | | | | Mean |
| | | | | Medi- | LS | Differ- |
| Subgroup | | N | Mean | an | Mean | ence |
| Overall | Placebo | 20 | 1.48 | 1.45 | 1.54 | |
| | Ampreloxetine | 20 | −0.05 | 0.05 | −0.03 | −1.57 |
| RW baseline OHSA | Placebo | 11 | 1.84 | 1.4 | 1.86 | |
| Composite <2.5 | Ampreloxetine | 9 | 0.98 | 0.4 | 0.95 | −0.90 |
| RW baseline OHSA | Placebo | 9 | 0.93 | 1.5 | 1.04 | |
| Composite >=2.5 | Ampreloxetine | 11 | −0.90 | −0.5 | −0.92 | −1.96 |
| Pre treatment | Placebo | 10 | 0.94 | 1.35 | 1.25 | |
| baseline OHSA | Ampreloxetine | 10 | −0.56 | −0.05 | −0.71 | −1.96 |
| Composite <5.3 | | | | | | |
| Pre treatment | Placebo | 10 | 1.92 | 2.1 | 1.73 | |
| baseline OHSA | Ampreloxetine | 10 | 0.46 | 0.3 | 0.76 | −0.97 |
| Composite >=5.3 | | | | | | |
| RW baseline | Placebo | 11 | 1.47 | 1.25 | 1.65 | |
| OHSA1 <=2 | Ampreloxetine | 10 | 0.86 | 0.25 | 0.74 | −0.91 |
| Pre treatment | Placebo | 9 | 1.5 | 1.6 | 1.22 | |
| baseline OHSA1 >2 | Ampreloxetine | 10 | −0.96 | −0.6 | −0.77 | −1.99 |
| Pre treatment | Placebo | 9 | 0.63 | 0.7 | 0.86 | |
| baseline OHSA1 <7 | Ampreloxetine | 9 | −0.11 | −0.1 | −0.22 | −1.08 |
| Pre treatment | Placebo | 11 | 2.03 | 2.1 | 1.73 | |
| baseline OHSA1 >=7 | Ampreloxetine | 11 | 0.00 | 0.5 | 0.41 | −1.31 |

It was also shown that at week 22 (the end of the 6-week double-blind randomized period), subjects with MSA who were administered ampreloxetine showed a decrease in the Anxiety Total Score, indicating effectiveness of ampreloxetine against anxiety associated with MSA and/or nOH. Table 12 shows changes in the Anxiety Total Score for MSA subjects at week 22 from baseline. The Anxiety Total Score was determined using standard procedures (see, e.g., Rishi et al. Hospital anxiety and depression scale assessment of 100 patients before and after using low vision care: A prospective study in a tertiary eye-care setting. Indian J Ophthalmol. 2017 November; 65(11):1203-1208.)

TABLE 12

Change from Baseline

| | Placebo | Ampreloxetine 10 mg |
|---|---|---|
| | (N = 64) | (N = 64) |
| N | 19 | 19 |
| Mean (SD) | 0.21 (1.873) | −1.05 (1.900) |
| Median | 0.00 | −1.00 |
| Q1, Q3 | −1.00, 2.00 | −3.00, 0.00 |
| Min, Max | −3.0, 4.0 | −3.0, 3.0 |
| LS Mean (SE) | 0.21 (0.405) | −1.05 (0.405) |
| LS Mean Difference (SE) | | −1.26 (0.573) |
| 95% CI for LS Mean | | (−2.43, −0.10) |
| Difference | | |
| P-value vs. Placebo | | 0.0343 |

Compared to MSA subjects at Week 4 shown in Table 5 (Example 6), ampreloxetine showed greater improvement against anxiety associated with MSA and/or nOH, suggesting that extended treatment (i.e. 4 weeks or more) surprisingly provides greater effectiveness. Similarly, ampreloxetine did not meet endpoints at Week 4 in Example 6, while extended administration of ampreloxetine showed clear improvement of nOH symptoms, as shown in Tables 9-11, and FIGS. 5 and 6. Accordingly, it is suggested that surprisingly, when administered for an extended period (i.e. 4 weeks or more), ampreloxetine shows efficacy which may not be shown when administered for a short period (i.e. less than 4 weeks).

### Example 8

Phase 3 Efficacy and Durability of Ampreloxetine for the Treatment of Symptomatic nOH in Subjects with Multiple System Atrophy

This is a Phase 3, multi-center, randomized withdrawal and long-term extension study to evaluate the sustained benefit in efficacy and durability of ampreloxetine (administered as ampreloxetine hydrochloride) in subjects with MSA and symptomatic nOH after 20 weeks of treatment. Eligible subjects are subjects with MSA and symptomatic nOH meeting all applicable study inclusion criteria and none of the applicable exclusion criteria.

The study consists of 4 periods: (i) up to 14 days of screening; (ii) 12-week open-label (OL) treatment with ampreloxetine, (ii) 8-week double-blind randomized placebo-controlled treatment, and (iii) 2 years long-term extension period.

### A. Study Population

This study enrolls adult subjects with MSA and confirmed symptomatic nOH who meet all of the applicable inclusion criteria and none of the applicable exclusion criteria defined below.

1. Inclusion Criteria

Subjects who meet the following applicable criteria are eligible for study enrollment.

(a) Subject is male or female and at least 30 years old.

(b) Subject has a diagnosis of possible or probable MSA of the Parkinsonian subtype (MSA-P) or cerebellar subtype (MSA-C) according to The Gilman Criteria (2008).

(c) Subject has a diagnosis of possible or probable MSA of the Parkinsonian subtype (MSA-P) or cerebellar subtype (MSA-C) confirmed by the Enrollment Steering Committee (ESC).

(d) Subject must meet the diagnostic criteria of nOH, as demonstrated by a sustained reduction in BP of ≥20 mmHg (systolic) or ≥10 mmHg (diastolic) within 3 min of standing as part of orthostatic standing test or being tilted up ≥60o from a supine position as determined by a tilt-table test.

(e) Subject must score ≤4 on UMSARS Part IV at Visit 1 (Screening).

(f) Subject must score at least a 4 on the OHSA item 1 at Visit 2 (Day 1).

(g) Subject must be willing to not take any prohibited medications during the study.

(h) If subject is female, the subject must not be pregnant, breastfeeding, or planning a pregnancy during the course of the study. A woman of childbearing potential must have a documented negative pregnancy test at screening. A woman is considered to be of childbearing potential unless she is postmenopausal (amenorrheic for at least 2 years) or documented to be surgically sterile (bilateral tubal ligation or total hysterectomy). A female subject may be admitted to the study on the basis of a negative urine pregnancy test. If the urine beta human chorionic gonadotropin (bHCG) test is positive, a serum bHCG test must be performed. The pregnancy test must be confirmed negative for a subject to be eligible for this study.

(i) During the study and for 30 days after receiving the last dose of the study drug, females of childbearing potential or males capable of fathering children must agree to use highly effective birth control measures (failure rate <1% when used consistently and correctly) or agree to abstain from sexual intercourse (Refer to Section 4.3).

(j) Subject is willing and able to provide signed and dated written informed consent to participate prior to initiation of any study related procedures.

(k) Subject is able to communicate well with the Investigator and clinic staff, understands the expectations of the study and is able to comply with the study procedures, requirements, and restrictions.

4. Exclusion Criteria

Subjects who meet any of the following criteria are not eligible for study enrollment.

(a) Subject has a systemic illness known to produce autonomic neuropathy, including, but not limited to, amyloidosis and autoimmune neuropathies. Subject with diabetes mellitus (DM) are evaluated on a case-by-case basis by the medical monitor and considered ineligible unless they meet all of the following criteria:
1. Well controlled type-2 DM in treatment with only oral medications and diet
2. HbA1C of ≤7.5% performed during screening or up to 12 weeks before screening
3. No clinically evident peripheral neuropathy (e.g., normal sensory examination on peripheral extremities)
4. No known retinopathy (e.g., annual ophthalmic exam is sufficient)
5. No nephropathy (e.g., absence of albuminuria and GFR >60).

(b) Subject has a known intolerance to other NRIs or SNRIs.

(c) Subject currently uses concomitant antihypertensive medication for the treatment of essential hypertension. (d) Subject has used strong CYP1A2 inhibitors or inducers within 7 days or 5 half-lives, whichever is longer, prior to Visit 2 (Day 1) or requires concomitant use until the Safety follow-up Visit.

(e) Subject has changed dose, frequency, or type of prescribed medication for orthostatic hypotension within 7 days prior to Visit 2 (Day 1). Midodrine and droxidopa (if applicable) must be tapered off and stopped at least 7 days prior to Visit 2 (Day 1). (f) Subject has known or suspected alcohol or substance abuse within the past 12 months (Diagnostic and Statistical Manual of Mental Disorders, Fourth Edition, Text Revision [DSM-IV-TR®] definition of alcohol or substance abuse). (e) Subject has clinically unstable coronary artery disease or had a major cardiovascular event (e.g., myocardial infarction) in the past 6 months.

(f) Subject has significant uncontrolled cardiac arrhythmia, history of complete heart block, or significant QTc prolongation (≥450 msec for males and ≥470 msec for females).

(g) Subject has a new onset of a neurological event (i.e., seizures, confusion, altered levels of consciousness, etc.) in the past 6 months.

(h) Subject has used any monoamine oxidase inhibitor (MAOI) within 14 days prior to Visit 2 (Day 1).

(i) Subject has a history of untreated closed angle glaucoma, or treated closed angle glaucoma that, in the opinion of an ophthalmologist, might result in an increased risk to the subject.

(j) Subject has a Montreal Cognitive Assessment (MoCA) <21.

(k) Subject is unable or unwilling to complete all protocol specified procedures including questionnaires.

(l) Subject has known congestive heart failure (New York Heart Association [NYHA] Class 3 or 4).

(m) Subject has had any malignant disease, other than carcinoma in situ of the cervix or basal cell carcinoma, within the past 2 years prior to Screening.

(n) Subject has a known gastrointestinal (GI) condition, which in the Investigator's judgment, may affect the absorption of study medication (e.g., ulcerative colitis, gastric bypass).

(o) Subject has psychiatric, neurological, or behavioral disorders that may interfere with the cognitive ability of the subject to give informed consent, understand and comply with study procedures, or interfere with the conduct of the study.

(p) Subject is currently receiving any investigational drug or has received an investigational drug within 30 days of dosing. An investigational drug is defined as a drug that is not approved by a regulatory agency (e.g., Food and Drug Administration [FDA]).

(q) Subject has a clinically significant abnormal laboratory finding(s) (e.g., alanine aminotransferase [ALT] or aspartate aminotransferase [AST] ≥3.0× upper limit of normal [ULN]; blood bilirubin [total] ≥3.0×ULN; estimated glomerular filtration rate (eGFR)<30 mL/min/1.73 m2, or any abnormal laboratory value that could interfere with safety of the subject).

(r) Subject has demonstrated lifetime suicidal ideation and/or suicidal behavior, as outlined by the C-SSRS (Baseline/Screening Version). Subject should be assessed by the rater for risk of suicide and the subject's appropriateness for inclusion in the study.

(s) Subject has a concurrent disease or condition (e.g., COVID-19), or recent surgery, that in the opinion of the Investigator, would confound or interfere with study participation or evaluation of safety, tolerability, or absorption of the study drug.

(t) Subject has known hypersensitivity to ampreloxetine (ampreloxetine hydrochloride), or any excipients in the formulation.

(u) Major surgery (i.e., procedures involving higher risk for infection and extended recovery period, such as, joint replacement, gastric bypass, open heart surgery, organ transplant, etc.) occurring less than 4 weeks prior to enrollment.

### B. Test Product, Dose, and Route of Administration: Regimen: Duration of Treatment

1. For OL Treatment Period

All subjects receive ampreloxetine and continue to take the study medication once daily starting on Day 2 through the end of the OL period. The test product is ampreloxetine supplied as a 10-mg tablet in 30 count high-density polyethylene bottles.

2. For RW Period

Subjects randomized to ampreloxetine receives ampreloxetine tablets and continue to take the study medication once daily starting in the morning following Visit 5 (Day 2 of RW period, Day 86) through the end of the RW period. The test product is ampreloxetine supplied as a 10-mg tablet in 30 count high-density polyethylene bottles labeled in a blinded fashion (i.e., bottle label for test product is indistinguishable from reference product except for a coded, unique bottle number).

3. For LTE Treatment Period

Subjects receives ampreloxetine and continue to take the study medication once daily starting on Day 142 through the end of the LTE period. The test product is ampreloxetine supplied as a 10-mg tablet in 30 count high-density polyethylene bottles.

4. For All Treatment Periods

Ampreloxetine is administered orally without regard to food at approximately the same time each morning and was taken with approximately 8 ounces (240 mL) of water.

### C. Reference Product, Dose, and Route of Administration: Regimen: Duration of Treatment

1. For OL Treatment Period

No subjects received the reference product during the OL treatment period.

2. For RW Period

Subjects randomized to PBO receive placebo tablets once daily starting in the morning following Visit 5 (Day 2 of RW period, Day 86) through the end of the RW period. The PBO tablets match ampreloxetine tablets in excipient content (except for absence of ampreloxetine), appearance, tablet count, and in packaging (i.e., bottle label for reference product is indistinguishable from test product except for a coded, unique bottle number).

3. For LTE Period

No subjects received the reference product during the LTE treatment period.

4. For All Treatment Periods

The PBO tablet was administered orally without regard to food at approximately the same time each morning and was taken with approximately 8 ounces of water.

### D. Study Execution

1. Screening

Subjects enters a screening period of up to 14 days to confirm eligibility. At Visit 1 (Screening), subjects provide a comprehensive medical history of their disease and treatments. The subjects' disease is characterized and documented by the principal investigator (PI) or sub-investigator. Subjects receive an assessment of their physical condition, including safety and laboratory evaluations and related aspects of their disease state. Eligible subjects undergo training on how to accurately score items of the OHQ Following the screening period (up to 14 days), subjects proceed to Visit 2 (Day 1) to further confirm the additional eligibility criteria prior to proceeding. This includes the completion of the OHQ in which a minimum score of 4 points in OHSA item 1 is required. Subjects meeting all inclusion criteria, none of the exclusion criteria, and whose disease characterization is confirmed by the independent Enrollment Steering Committee (ESC) receives ampreloxetine in the OL period.

2. Open Label Period (Weeks 1 to 12)

Beginning on Day 2 (the morning following Visit 2), subjects receive a single dose of OL 10 mg ampreloxetine once daily and continue thereafter for the 12-week duration of the OL period.

Subjects enrolled into the OL period of the study have visits scheduled for Day 29 (Visit 3, Week 4), Day 57 (Visit 4, Week 8) and Day 85 (Visit 5, Week 12) as outlined in the Schedule of Study Procedures (Table 1). At Visit 4, following 8 weeks of OL treatment, subjects must demonstrate a reduction in OHSA item 1 of at least 2 points compared to the baseline value, (i.e., as measured at Visit 2), in order to continue in the study.

All subjects meeting the continuation criterion at Visit 4 (Week 8) continue receiving OL ampreloxetine tablets for 4 additional weeks (12 weeks total).

Those subjects not meeting this continuation criterion at Visit 4 must be discontinued and undergo Early Termination Visit within 3 days of last dose. The Safety Follow-up Visit (End of Study) must be completed 30 days (±7 days) from the date of the last dose and may be conducted in-clinic or by telephone.

3. Double-Blind Randomized Withdrawal Period (Weeks 13 to 20)

Following the completion of a total of 12 weeks OL treatment with ampreloxetine subjects are assessed for randomization in a 1:1 manner at Visit 5. Only subjects with OHSA item 1 score of ≤7 are eligible for randomization for the double-blind treatment period.

Eligible subjects receive 8 weeks of double-blind treatment of 10-mg ampreloxetine or placebo once daily.

There is a cap for randomization (Visit 5) on subjects with an UMSARS Part IV score of 4 at Screening (Visit 1) once it reaches 25% (i.e., 18 randomized subjects).

Subjects who are not eligible for randomization must be discontinued and undergo Early Termination Visit within 3 days of last dose. The Safety Follow-up Visit (End of Study Visit) must be completed 30 days (±7 days) from the date of the last dose and may be conducted in-clinic or by telephone.

4. Treatment Period (Weeks 1 to 22)

No dose reduction is permitted during the treatment period. Subjects unable to tolerate 10-mg ampreloxetine are discontinued from the study. At any time during the study, if a subject meets at least one of the following stopping rules, they should be discontinued and undergo an Early Termination Visit and a Safety Follow-up Visit (End of Study Visit):

(a) A determination from the PI that further administration of the investigational product may pose a safety concern to the subject

(b) Sustained (at least 4 hours) SBP ≥180 mmHg or diastolic BP (DBP) ≥110 mmHg after 3 min of standing or after 5 min in the sitting position, or a sustained (at least 4 hours) SBP ≥180 mmHg or DBP ≥110 mmHg measured in the supine state (head/torso elevated at approximately 30o from horizontal position).

(c) Intolerable AE as determined by the Investigator

(d) Female subject becomes pregnant

Safety assessments include a physical examination, neurological examination, vital signs (body temperature, HR, respiratory rate [RR], and BP), body weight, 12-lead ECGs, laboratory tests (hematology, chemistry, and urinalysis), Columbia Suicide Severity Rating Scale (C-SSRS) and monitoring of AEs.

Subjects are requested to refrain from making any significant dietary changes throughout the duration of the study. During their scheduled visits, subjects should be reminded to maintain an adequate fluid intake.

For those subjects who do not complete the 8-week double-blind RW period, they complete the Early Termination Visit within 3 days from the date of the last dose. For all subjects, the Safety Follow-up Visit (End of Study Visit) must be completed 30 days (±7 days) from the date of the last dose and may be conducted in-clinic or by telephone. Subjects completing the 8-week double-blind RW period are eligible to continue into the 2-year LTE period.

5. Long-Term Treatment Extension

During LTE, subjects receive 10-mg ampreloxetine once daily and undergo study visits consistent either with the standard of care for a subject outside of a clinical trial or at a minimum an in-clinic visit once per year (Table 1).

All sites are allowed at Investigator discretion to conduct either in-clinic or remote unscheduled visit(s) for subject safety or unexpected subject medical needs outside of the regular visit schedule. Data collected during these visits may include any protocol-specified assessments which are captured in the clinical database.

The Safety Follow-up Visit (End of Study) must be completed 30 days (±7 days) from the date of the last dose and may be conducted in-clinic or by telephone.

### E. Study Evaluations

The efficacy assessments for this study includes: OHQ, OST (Orthostatic Standing Test) inclusive of 3 minute standing test, and pharmacokinetic assessments.

Exploratory assessments includes: Hospital Anxiety and Depression Scale (HADS), Patient Global Impression of Severity-Symptom (PGIS-Symptom), Patient Global Impression of Severity-Activity (PGIS-Activity), Patient Global Impression of Change-Symptom (PGIC-Symptom), Patient Global Impression of Change-Activity (PGIC-Activity).

Safety and tolerability assessments for this study included: C-SSRS; AEs; medication and medical history, physical examination; neurological examination; height and weight; vital signs, ECGs; laboratory tests, hematology, chemistry, urinalysis, dosing diary dispensation and/or collection; and unscheduled visits.

### F. Statistical Methods

1. Sample Size

A total sample size of 64 would provide an overall power of greater than 90% to detect a difference of 1.5 points with a standard deviation of 1.8 points at two-sided alpha level of 0.05 for the primary endpoint of the change in OHSA composite score at Week 8 (Visit 9, Day 141) during the double-blind RW period. The power calculation includes a single interim analysis for efficacy on approximately 56% of subjects where a Hwang-Shih-DeCani alpha-spending function with the gamma parameter (−3.5) is constructed to implement group sequential boundaries that control the type I error rate. Accounting for a 10% drop out rate during the RW period, a sample size of 72 would be required at randomization (Visit 5).

Assuming that 70% are eligible for randomization at the end of the 12-week OL period, approximately 102 subjects would be enrolled to ensure 72 subjects are expected to continue into the randomized treatment period.

2. Study Endpoints

The primary study endpoint is the change in OHSA composite score at Week 8 (Visit 9, Day 141) during the double-blind RW period. The assessments done at the Week 12 (Visit 5, Day 85) visit in the OL period prior to randomization are considered baseline for the double-blind RW period of the study.

The secondary endpoints are:

(a) Change from baseline in OHDAS item 1 (activities that require standing for a short time) at Week 8 post randomization (Visit 9, Day 141)

(b) Change from baseline in OHDAS item 3 (activities that require walking for a short time) at Week 8 post randomization (Visit 9, Day 141)

The exploratory endpoints include:

(c) Change from baseline in 3 min standing SBP during the OST at Week 8 post randomization (Visit 9, Day 141)

(d) Change from baseline in OHQ composite score at Week 8 post randomization (Visit 9, Day 141)

(e) Change from baseline in the short time daily activities composite score (OHDAS item 1 and OHDAS item 3) at Week 8 post randomization (Visit 9, Day 141); The short time daily activities composite score is defined as the average of the OHDAS item 1 and OHDAS item 3 item scores from the OHQ questionnaire.

(f) Change from baseline in HADS anxiety domain at Week 8 post randomization (Visit 9, Day 141)

(g) Change from baseline in HADS depression domain at Week 8 post randomization (Visit 9, Day 141)

(h) PGIS-Symptom and PGIS-Activity at baseline and at Week 8 post randomization (Visit 9, Day 141)

(i) Change from baseline in PGIS-Symptom and PGIS-Activity at Week 8 post randomization (Visit 9, Day 141)

(j) PGIC-Symptom and PGIC-Activity at Week 8 post randomization (Visit 9, Day 141)

3. Endpoint Analysis

The primary estimand addresses the following clinical question of interest: the mean difference in the change in OHSA composite score at Week 8 during the double-blind RW period in subjects with MSA and symptomatic nOH treated with ampreloxetine vs. placebo regardless of discontinuation of investigational intervention for any reason and regardless of initiation of rescue medication.

The primary estimand follows a treatment policy strategy and is described by the following attributes:

(a) Population: Subjects with MSA and symptomatic nOH (i.e., FAS in double-blind RW period).

(b) Endpoint: Change in OHSA composite score at Week 8 during the double-blind RW period.

(c) Treatment condition: The investigational interventions regardless of discontinuation for any reason, with or without rescue medication (treatment policy strategy).

(d) Population-level summary: Difference in mean changes between treatment conditions.

The primary estimand is analyzed using mixed model for repeated measures (MMRM) to compare treatment differences based on the FAS. The model includes fixed effect class terms of treatment, UMSARS Part IV at Visit 1 (<4, 4) and visit, a continuous covariate of baseline score (Visit 5, Day 85) and a random subject effect with an unstructured covariance structure. Interaction terms for visit*treatment and visit*baseline are also included. If the model doesn't converge, compound symmetry or other covariance structures are used as alternative covariance structure. Least-square means and 95% confidence intervals on the differences between ampreloxetine and placebo are be calculated and presented. Missing data is assumed missing at random (MAR).

Sensitivity analyses on the primary analysis are carried out using a missing not at random approach to missing data through multiple imputation. Supplementary estimand strategies are specified in the Statistical Analysis Plan (SAP).

The primary analysis is repeated on a set of prespecified subgroups and presented in graphical format. Details are specified in the SAP.

Individual components of the OHSA composite score are presented as a supportive analysis and are analyzed using a similar mixed model for repeated measures (MMRM) as outlined for the primary analysis.

There are four key secondary estimands of interest that follow a treatment policy strategy and have the same treatment condition, remaining intercurrent events and population-level summary attributes as the primary endpoint. The population and estimand attributes are as follows:

Key Secondary Estimand 1

(a) Population: Subjects with MSA and symptomatic nOH and UMSARS Part IV <4 at Visit 1

(b) Endpoint: Change from baseline in symptom impact on activities requiring a short time standing (OHDAS item 1) at Week 8 post randomization (Visit 9, Day 141)

Key Secondary Estimand 2

(a) Population: Subjects with symptomatic nOH due to MSA

(b) Endpoint: Change from baseline in symptom impact on activities requiring a short time standing (OHDAS item 1) at Week 8 post randomization (Visit 9, Day 141)

Key Secondary Estimand 3

(a) Population: Subjects with symptomatic nOH due to MSA and UMSARS Part IV <4 at Visit 1

(b) Endpoint: Change from baseline in symptom impact on activities requiring a short time walking (OHDAS item 3) at Week 8 post randomization (Visit 9, Day 141)

Key Secondary Estimand 4

(a) Population: Subjects with symptomatic nOH due to MSA

(b) Endpoint: Change from baseline in symptom impact on activities requiring a short time walking (OHDAS item 3) at Week 8 post randomization (Visit 9, Day 141)

Secondary efficacy endpoints are analyzed using MMRM to compare treatment differences. The model in the UMSARS Part IV <4 subpopulation analysis includes fixed effect class terms of treatment, visit, and continuous covariate of baseline score of the respective measure, a random subject effect, with an unstructured covariance structure. Interaction terms for visit*treatment and visit*baseline are be included. If the model doesn't converge, compound symmetry or other covariance structures are used as alternative covariance structure. Least-square means and 95% confidence intervals on the differences between ampreloxetine and placebo are calculated and presented. The analysis model in the full population also includes a fixed effect class term of UMSARS Part IV at Visit 1 (<4, 4).

Exploratory endpoints involving assessment of change from baseline at multiple time points such as the OHQ composite score are analyzed similarly to the primary analysis.

For exploratory endpoints involving assessment of change from baseline but only at Week 8 post randomization (Visit 9, Day 141) such as HADS domains, an analysis of covariance (ANCOVA) is used to compare treatment differences based on the FAS. The model includes fixed effect of treatment and continuous covariate of baseline score of the respective scales.

The analysis of the PGIS and PGIC to support the interpretation of the primary and secondary endpoints are detailed in the SAP.

In addition, descriptive analysis of efficacy data collected during OL period is carried out for all subjects.

While the subject matter of the present disclosure has been described with reference to specific aspects or embodiments thereof, it will be understood by those of ordinary skill in the art that various changes can be made or equivalents can be substituted without departing from the true spirit and scope of the disclosure. Additionally, to the extent permitted by applicable patent statutes and regulations, all publications, patents and patent applications cited herein are hereby incorporated by reference in their entirety to the same extent as if each document had been individually incorporated by reference herein.

What is claimed is:

1. A method for treating symptomatic neurogenic orthostatic hypotension (nOH) in a subject having multiple system atrophy (MSA), the method comprising administering daily to the subject for at least about 8 weeks a pharmaceutical composition comprising a pharmaceutically acceptable carrier and about 10 mg (free base equivalents) of ampreloxetine or a pharmaceutically acceptable salt thereof, wherein the administration results in reduction of at least one of Orthostatic Hypotension Symptom Assessment (OHSA) composite score, Orthostatic Hypotension Daily Activities Scale (OHDAS) item 1 score (standing for a short time), and OHDAS item 3 score (walking for a short time).

2. The method of claim 1, wherein the pharmaceutical composition is administered to the subject orally.

3. The method of claim 1, wherein the pharmaceutical composition is administered to the subject once per day.

4. The method of claim 1, wherein the subject being treated experiences symptoms of neurogenic orthostatic hypotension in the absence of treatment with the pharmaceutical composition as determined using the Orthostatic Hypotension Assessment Scale (OHSA).

5. The method of claim 1, wherein the subject has MSA subtype P (MSA-P).

6. The method of claim 1, wherein the subject has MSA subtype C (MSA-C).

7. The method of claim 1, wherein prior to treating, the subject has a sustained reduction in BP of ≥20 mmHg (systolic) or ≥10 mmHg (diastolic) within 3 min of standing as part of orthostatic standing test or being tilted up ≥60° from a supine position as determined by a tilt-table test.

8. The method of claim 1, wherein prior to treating, the subject has score 4 or less on Unified Multiple System Atrophy Rating Scale (UMSARS) Part IV.

9. The method of claim 1, wherein prior to treating, the subject has score of 4 on Unified Multiple System Atrophy Rating Scale (UMSARS) Part IV.

10. The method of claim 1, wherein the subject has at least a 4 on the OHSA item 1 score before the administration.

11. The method of claim 1, wherein the pharmaceutical composition is administered for at least about 12 weeks.

12. The method of claim 1, wherein ampreloxetine is administered as a hydrochloride salt.

13. The method of claim 1, wherein the pharmaceutically acceptable carrier comprises one or more of microcrystalline cellulose, lactose and magnesium stearate.

14. A method for treating symptomatic neurogenic orthostatic hypotension (nOH) in a subject having multiple system atrophy (MSA), the method comprising administering daily to the subject for at least about 22 weeks a pharmaceutical composition comprising a pharmaceutically acceptable carrier and about 10 mg (free base equivalents) of ampreloxetine or a pharmaceutically acceptable salt thereof, wherein:
(a) the subject being treated experiences symptoms of neurogenic orthostatic hypotension in the absence of treatment with the pharmaceutical composition as determined using the Orthostatic Hypotension Assessment Scale (OHSA); and
(b) administration of the pharmaceutical composition daily to the subject for at least about 22 weeks results in a measurable reduction in at least one of (i) the subject's OHSA composite score, (ii) Orthostatic Hypotension Questionnaire (OHQ) composite score, (iii) Orthostatic Hypotension Daily Activity Scale (OHDAS) composite score, or (iv) OHDAS item 1 (standing short time).

15. The method of claim 14, wherein the pharmaceutical composition is administered to the subject orally.

16. The method of claim 14, wherein the pharmaceutical composition is administered to the subject once per day.

17. The method of claim 14, wherein the supine plasma norepinephrine level in the subject is less than about 350 pg/mL before treatment with the pharmaceutical composition.

18. The method of claim 14, wherein the supine plasma norepinephrine level in the subject is greater than about 500 pg/mL after about 8 weeks of treatment with the pharmaceutical composition.

19. The method of claim 14, wherein the subject has MSA subtype P (MSA-P).

20. The method of claim 14, wherein the subject has been diagnosed with MSA for at least 1.3 years before the administration.

21. The method of claim 14, wherein the subject had onset of nOH 1.6 years or more before the administration.

22. The method of claim 14, wherein the subject had an OSHA composite score of 5 or greater before the administration.

23. The method of claim 14, wherein the subject had an OSHA item 1 score of 7 or greater before the administration.

24. The method of claim 14, wherein the subject has a score ≤4 on Unified Multiple System Atrophy Rating Scale (UMSARS) Part IV before the administration.

25. The method of claim 14, wherein the subject has a score of 4 on Unified Multiple System Atrophy Rating Scale (UMSARS) Part IV before the administration.

26. The method of claim 14, wherein ampreloxetine is administered as a hydrochloride salt.

27. The method of claim 14, wherein the pharmaceutical composition is administered for at least about 12 months.

28. The method of claim 14, wherein the pharmaceutically acceptable carrier comprises one or more of microcrystalline cellulose, lactose and magnesium stearate.

29. A method of treating symptomatic neurogenic orthostatic hypotension (nOH) in a subject having multiple system atrophy (MSA), comprising:
administering the subject for at least 8 weeks with a pharmaceutical composition comprising a pharmaceutically acceptable carrier and about 10 mg (free base equivalents) of ampreloxetine or a pharmaceutically acceptable salt thereof.

30. The method of claim 29, further comprising, if the subject is responsive to ampreloxetine or a pharmaceutically acceptable salt thereof, further administering the pharmaceutical composition to the subject.

31. The method of claim 29, wherein the pharmaceutical composition is administered for at least 12 weeks.

32. The method of claim 29, further comprising determining whether the subject shows decrease of at least 2 points in Orthostatic Hypotension Symptom Assessment (OHSA) item 1 score subsequent to the administration

33. The method of claim 29, wherein the pharmaceutical composition is administered to the subject orally.

34. The method of claim 29, wherein the pharmaceutical composition is administered to the subject once per day.

35. The method of claim 29, wherein the MSA is MSA subtype P (MSA-P).

36. The method of claim 29, wherein the MSA is MSA subtype P (MSA-C).

37. A method of treating symptomatic neurogenic orthostatic hypotension (nOH) in a subject having multiple system atrophy (MSA), comprising administering to the subject a pharmaceutical composition comprising a pharmaceutically acceptable carrier and ampreloxetine or a pharmaceutically acceptable salt thereof, wherein the subject has:
(a) Orthostatic Hypotension Assessment Scale (OSHA) composite score of 5 or greater before the administration; and/or
(b) OSHA item 1 score of 7 or greater before the administration.
